(19) United States
(12) Patent Application Publication
(10) Pub. No.: US 2008/0021391 A1
(43) Pub. Date: Jan. 24, 2008

(54) Syringe

(75) Inventors: John Polidoro, Manchester, CT (US); Spencer Kinsey, Manchester, CT (US); Chad Smutney, Manchester, CT (US)

Correspondence Address: NIXON & VANDERHYE, PC, 901 NORTH GLEBE ROAD, 11TH FLOOR, ARLINGTON, VA, 22203, US

(73) Assignee: ID-Tech-Limited, Darras Hall (GB)

(21) Appl. No.: 11/826,690

(22) Filed: Jul. 17, 2007

Related U.S. Application Data
(60) Provisional application No. 60/831,211, filed on Jul. 17, 2006.

(51) Int. Cl. A61M 5/50

(52) U.S. Cl. 604/110

(57) ABSTRACT

An auto-disable syringe (50,150) is disclosed which prevents re-use of the syringe whatever dose is delivered on the first use. A restrictor bobbin comprises an element (1,100) which spans the barrel of the syringe and which, after initial distal movement of the plunger (52, 152) rotates and wedges in the barrel when the plunger is moved proximally. The wedged restrictor bobbin (1, 100) prevents further substantial proximal movement of the plunger (52, 152) whilst allowing further distal movement. A small degree of repeated proximal and distal movement is permitted to allow for an aspiration step.

### TECHNICAL FIELD

The present invention relates to single use syringes, that is to say syringes which are intended to be used once and which are adapted in some way to prevent or at least to hinder further use.

### BACKGROUND ART

Disposable syringes are known in which a cylindrical barrel formed of transparent plastics material receives a piston which is slidable within the barrel. A shaft, which may be of cruciform or other, e.g. circular, section extends from the piston to a plunger handle for enabling the piston to be displaced along the barrel in a first or proximal direction to cause injectable fluid or body fluid to be drawn into the barrel via an aperture at one end of the barrel, or in a second or distal direction to cause the fluid to be expelled out of the aperture or to be injected into a patient via a needle.

Syringes of this type are generally sold as disposable items and are intended to be used only once to negate the risk of transmission of diseases between patients. However, such syringes suffer from the drawback that it is difficult to prevent such syringes from being re-used, which re-use increases the risk of transmission of serious, life-threatening, conditions such as certain bacterial infections, viral hepatitis, and HIV.

Numerous designs have been proposed for syringes which are intended to negate or reduce the risk of the syringe being re-used. However, there are considerable challenges involed in designing a syringe which meets all desiderata, including, without limitation:
(a) allowing aspiration or flashback of blood to check for correct location of the needle in a patient;
(b) ability to deliver variable doses;
(c) smooth operation;
(d) simplicity of manufacture and use
(e) ability to prevent drawing up and subsequent delivery of a number of doses of medicament whose combined volume is less than or equal to the total syringe volume.

EP0925083B1 discloses a single use syringe comprising a barrel with an internal annular groove at the proximal end and a further annular groove near the distal end but spaced from it. The plunger is formed with an integrally moulded barb-like flange adjacent the head, which flange bears resiliently against the barrel interior wall. The flange is able to move unrestricted in either proximal or distal direction along the majority of the barrel interior wall; however, the flange is only able to pass the annular grooves in the distal direction. The syringe is supplied with the plunger not fully depressed, so that the restrictor flange is on the proximal side of the more distal of the two grooves. Liquid may be drawn up into the syringe until the flange encounters the proximal groove, which prevents the plunger being withdrawn completely from the barrel. Liquid may be discharged freely from the syringe by depressing the plunger, the flange passing just beyond the more distal of the grooves when the plunger head is moved to the extreme distal end of the barrel. In this position, the plunger is now prevented from being withdrawn again because the flange will not pass the groove in the barrel. This design is elegant and simple but suffers from a number of drawbacks, not least the fact that there will inevitably be a slight jolt as the flange passes the more distal of the grooves when an injection is being given, and this jolt is likely to be felt by a patient. Furthermore, because of the unrestricted movement of the plunger between the two grooves, the syringe could potentially be used again and again provided the plunger is never fully depressed. After unpacking a syringe, it is normal to cycle the plunger over a short distance to check that the plunger is free to move and, if it is not, to free it: sometimes there can be a degree of adhesion between the plunger head and the barrel due to the length of time of storage, or due to the effects of gamma sterilisation. This is particularly the case with plunger heads which have had silicone lubricant applied to them. During this movement it would be relatively easy to lock the plunger of this syringe by moving the flange past the distal groove. This design is the only one of which the inventors are currently aware which properly can be used to aspirate a flash of blood for checking needle position in a patient prior to injecting a drug.

U.S. Pat. No. 5,000,737 discloses a syringe having a single piece metal barbed restrictor element located between the plunger shaft and the cylindrical interior syringe barrel wall. The element has barbs facing towards the plunger which prevent movement of the plunger distally with respect to the element, and barbs facing the barrel which prevent movement of the element proximally with respect to the barrel. The restrictor element is initially located near the proximal end of the barrel; thus initial proximal movement of the plunger to draw up liquid is permitted as the plunger can slide past the restrictor in this direction. Subsequent depression of the plunger to deliver liquid is permitted because the restrictor can move distally with respect to the barrel, and hence when the plunger is depressed it carries the element with it. Further movement is of course prevented. This design has many similarities with some of the embodiments set out below; however, it does not permit aspiration of a flash of blood nor repeated movement to assist in reconstitution of lyophilised drug.

U.S.2003/0060759 discloses a design which has similarities to that of U.S. Pat. No. 5,000,737, but also some important differences. It, too, utilises a single piece metal barbed restrictor element mounted between the plunger shaft and the barrel interior wall, and it employes outwardly facing barbs to restrict the motion of the element with respect to the barrel. In this design, however, the plunger shaft has a stepped form with a shoulder part way along it. The restrictor element has a spring tang which acts against the barrel and forces it against the plunger shaft. The restrictor starts out at the proximal end of the shaft; withdrawal of the plunger past the restrictor is permitted until an enlarged diameter portion of the shaft, near the plunger head, comes into engagement with the restrictor. At the same time, the proximal end of the restrictor snaps behind the shoulder on the plunger; thereby movement of the plunger in either direction with respect to the restrictor is prohibited. The plunger can be depressed, carrying the restrictor with it to the distal end of the barrel, and then the plunger is incapable of further movement. This design is simple and has been used in a commercial vaccination syringe product. It suffers from the disadvantage that it may be used repeatedly, provided the user does not draw up the plunger to the point where the restrictor snaps into place on the reduced diameter part of the plunger shaft.

Furthermore, once the restrictor has locked into place, which is of course the intention, aspiration of a flash of blood is not possible.

U.S. Pat. No. 5,222,942 discloses a design based on a ratchet system. A collar is installed in an initial distal position between plunger shaft and barrel. The plunger shaft is formed with annular ratchet teeth, and corresponding teeth are formed on the collar. The ratchet does not permit the plunger to be moved proximally past the collar, so when the plunger is initially drawn back in order to draw up liquid, it carries the collar with it to the extreme proximal end of the barrel where a formation on the barrel prevents the collar and plunger from being withdrawn completely from the barrel. The ratchet is such that the plunger may then be depressed past the collar to dispense liquid, and then of course the syringe is disabled. This design does not allow for aspiration of a flash of blood.

WO2005/058397A1 ('397), the contents of which are incorporated herein by reference, discloses a number of designs which are intended to allow for taking a flash of blood and possibly to allow for reconstitution of powdered drug. The designs described in '397 are intended to be simple and inexpensive and to have a smooth action which will not give rise to uncomfortable jolts when a patient is receiving an injection. The application is assigned to the assignee of the present application.

The designs described in '397 work on the principle of providing for a small amount of “lost motion” between a restrictor element and the syringe plunger, which allows for unlimited repeated distal and proximal movement over a distance which is just sufficient for the purpose of obtaining a flash of blood (or optionally for reconstitution of powdered drug). The distance may be carefully chosen so that it is sufficiently small to prevent, hinder or discourage repeated use of the syringe to deliver small doses of drug.

As stated above, achievement of all desiderata for a non reusable syringe in a single design is difficult, and there is normally a trade off between the complexity of the design and the the number of these goals which is achieved by the design. One potential issue with the designs described in '357 is that they will allow for drawing up and delivery of a volume of drug which is smaller than the usable volume of the syringe, and then drawing up and delivery of a further small dose or doses until the total volume of the syringe has been used. For example, a 5 ml syringe may be used to draw up 3 ml of liquid drug and then administer it. At this point the restrictor element has been moved a corresponding distance along the plunger or barrel, but there remains a distance corresponding to 2 ml of syringe volume over which the restrictor may move before the syringe is fully disabled; therefore a further drawing up and delivery of a 2 ml dose of medicament is possible before the syringe is fully disabled.

### DEFINITIONS

Throughout this specification, the terms “distal” and “proximal” will be interpreted with respect to the user of the syringe, i.e. the person administering an injection. Thus the “proximal” end of the syringe is the open end into which the plunger is received, and the “distal” end is the nozzle/needle end.

The terms “usable length” and “usable extent” as used herein with respect to a syringe barrel mean that portion of the barrel's length over which the plunger head is intended to be able to travel in the course of normal use, that is to say in the course of drawing up and discharging/injecting fluid. In some cases this can be a relatively small proportion of the overall length of the syringe, e.g. if it is desired that a restrictor bobbin be inserted deep into the barel so as to make it harder for it to be removed by a user who may wish to deactivate the single use feature of the syringe.

The term “movement” as used herein, unless stated to the contrary, refers to movement substantially along the axis of the syringe, that is to say along the length of the syringe. Similarly the term “direction”, as used herein with regard to the movement of components, refers to one or the other direction along the axis of the syringe, i.e. the proximal sense or the distal sense.

The terms “restricted” and “restrict” as used herein with respect to movement of a component of the syringe with respect to another component are intended to mean that a degree of restriction of movement is provided which is appropriate for the particular syringe. What is important is that the overall design of the syringe is such that a user attempting to circumvent its non-reuse features is prevented from doing so or is at least severely hampered. Different degrees of “restriction” may be required for different designs. In modified versions of some of the embodiments described herein, the plunger may have a weak point and be designed to break if a user tries to move the plunger in a restricted direction, thereby rendering the syringe inoperable. In a syringe incorporating such a feature, the force needed to move syringe components in a “restricted” direction may not be very great, e.g. 30-100N, provided the plunger is designed to break when a force lower than this is applied. A syringe in which the plunger did not have such a weak point may require that a greater force is able to be resisted.

### SUMMARY OF THE INVENTION

According to a first aspect of the invention, a syringe comprises:
(a) a plunger including a plunger head and a shaft;
(b) a barrel;
(c) a restrictor bobbin engaged with the plunger shaft so as to limit movement of the bobbin with respect to the plunger shaft in a predetermined direction;
(d) the restrictor bobbin having a outer barb, tine, serration, edge or the like engaged with an interior surface of the barrel;
(e) the restrictor bobbin, or a first part thereof which, preferably, substantially spans the barrel, being rotatable within the barrel;
(f) whereby movement of the plunger in the predetermined direction relative to the barrel causes the restrictor bobbin, or said first part thereof, to rotate and wedge in the barrel.

The restrictor bobbin may, alternatively or in addition, wedge between the barrel and the plunger.

The restrictor bobbin preferably spans the barrel.

The said predetermined direction is preferably the proximal direction, i.e. away from the syringe nozzle. In this event, preferably, the outer barb, tine, serration, edge or the like is oriented such as to limit distal movement of the bobbin with respect to the barrel.

The bobbin may include an inner edge, tine, barb, serration or the like which is engaged with the syringe plunger whereby said limitation of movement of bobbin with respect to plunger in the predetermined direction is provided. In this arrangement, the said inner edge, tine, barb, serration or the like may be adapted to engage with an unmodified plunger, i.e. one which has no features designed to work with an auto-disable system. Normally such a plunger would be substantially uniform along most of its length e.g. 70% of its length, preferably 80%, more preferably 90% of its length. This arrangement has the advantages that (i) it allows the system to work with an unmodified standard plunger and (ii) it allows the restrictor bobbin to function at substantially any relative position of plunger and barrel. Alternatively, the bobbin and plunger may be provided with cooperating features, such as ratchet features, for the purpose of limiting the movement between plunger and bobbin.

The restrictor bobbin may comprise two separate parts: a first part as described above including the outer barb, tine, serration edge, etc and a second part which includes the plunger engaging feature described above.

The said first and second parts may include cooperating formations which permit relative sliding of the said first and second parts over a limited distance which is less than the usable extent of the syringe (preferably between 1 and 50%, more preferably between 1 and 25%, still more preferably between 5 and 15% of the usable extent of the syringe).

Rotation of the bobbin, or first part thereof, may not only act to wedge it in the barrel but also may provide a degree of lost motion such that the plunger may be cycled repeatedly over a relatively small distance as the bobbin, or first part thereof, rotates back and forth. The said relatively small distance may be between 1 and 50%, more preferably between 1 and 25%, still more preferably between 1 and 10% of the usable extent of the syringe.

The said first part of the restrictor bobbin, or the bobbin itself if it is a single component design, may have a second plunger wedging edge, tine, barb, serration or the like adapted to engage with the plunger on rotation of the bobbin or first part thereof in order prevent or hinder movement of the plunger.

Rotation of the bobbin or first part thereof may be inhibited at one or more stages in the cycle of operation of the syringe. For example, the bobbin may be located at the proximal end of the usable extent of the barrel, adjacent a reduced diameter portion or undercut in the barrel wall, such that the undercut prevents rotation and thus wedging of the bobbin, or first part thereof, when the plunger is moved in a proximal direction. Movement of the plunger in a distal direction may cause the bobbin to move distally away from the undercut. Thereafter, proximal plunger movement may rotate and wedge the bobbin, or first part thereof, in the barrel (and optionally also against the plunger) since the undercut is not preventing such rotation. The reduced diameter portion (undercut) of the syringe may be located some way down the barrel rather than at the proximal end, if it is desired to make the restrictor bobbin less accessible to help prevent tampering. The undercut would still in this case represent the limit of the usable length/volume of the barrel.

In one embodiment of the invention, therefore, the syringe is provided with an undercut or reduced diameter portion at or near its proximal end, or at the proximal end of its usable volume, which acts to prevent or hinder rotation of the bobbin or of said part thereof being caused by proximal movement of the plunger, when the bobbin or said part thereof is located adjacent the said undercut. Such undercuts are standard features on most syringes.

In another aspect of the invention a syringe comprises:
a plunger,
a barrel,
a restrictor bobbin, wherein the restrictor bobbin includes:-
   a barrel locking formation or formations (e.g. an edge, barb, tine or serration) in engagement with the interior surface of the barrel and arranged to permit only unidirectional movement with respect to the barrel,
   any such barrel locking formation or formations being located on a first side only of the bobbin,
   a plunger locking formation (e.g. an edge, barb, tine, serration) arranged to be brought into or out of engagement with the plunger by rotation of the bobbin.

In yet another aspect of the invention a syringe comprises:
a plunger,
a barrel,
a restrictor bobbin, wherein the restrictor bobbin includes:-
   an arch formation mounted over the syringe plunger,
   said arch formation and plunger being configured to allow limited relative rotation between the formation and the plunger,
   said arch formation having on only one side thereof a barrel locking formation or formations (e.g. an edge, barb, tine or serration) in engagement with the interior surface of the barrel and arranged to permit only unidirectional movement with respect to the barrel,
   an edge of said arch formation being engageable with the plunger on rotation of the bobbin thereby to restrict relative movement of the plunger and bobbin in a predetermined direction.

The restrictor bobbin may be of unitary single part design, having a plunger wedging edge, tine, barb, serration or the like as described above. In this event, the bobbin may also be provided with a second plunger engaging edge, tine, barb, serration or the like adapted to resist or hinder movement of the plunger relative to the bobbin in the opposite direction to that which is resisted by the said plunger wedging edge, tine, barb, serration or the like. Preferably, the bobbin wedges to prevent, resist or hinder proximal movement of the plunger with respect to the bobbin and also proximal movement of the bobbin with respect to the barrel, whilst the said second plunger engaging edge, tine, barb, serration or the like is adapted to prevent, resist or hinder distal movement of the plunger relative to the bobbin.

According to the invention a restrictor element or bobbin for use in an auto-disable syringe comprises a shaped plate, the plate having a central U-shaped portion adapted to be located on a syringe plunger shaft and first and second flanges extending from opposite sides of the central U-shaped portion, the element having a proximal and distal end and having:-
(a) an outer barb, tine or serration located on the said first flange at an edge remote from the central U-shaped portion and oriented such as to resist movement of the element in a proximal direction with respect to a syringe barrel in which it is installed, by engaging a wall of the said barrel
(b) an inner barb, tine or serration located on the second flange and oriented such as to resist movement of the element in a proximal direction with respect to a syringe plunger shaft on which it is installed;
(c) the second flange of the restrictor element being adapted not to substantially resist movement when engaged with the said barrel wall.

In another aspect, a restrictor element or bobbin according to the invention for use in an auto-disable syringe may comprise:
A main arched body adapted to be mounted on a plunger of the syringe,
An outwardly extending barb, tine or serration for engaging with a barrel of the syringe such as to resist movement of the bobbin in a predetermined direction, e.g. the proximal direction,
Any such outwardly directed formation or formations being located on one side only of the bobbin,
An inwardly directed barb, tine or serration for engaging with the plunger of the syringe such as to resist movement of the bobbin in a predetermined direction, e.g. the proximal direction, with respect to the plunger.

According to the invention, a method of delivering medication or using an auto-disable syringe is provided, where the syringe comprises a barrel, a plunger having a plunger head and a plunger shaft, and a restrictor element or bobbin located between the plunger shaft and barrel, and the method comprises:
(a) if not already fitted, fitting a hypodermic needle
(b) drawing up a liquid medicament into the syringe by withdrawing the plunger proximally;
(c) inserting the hypodermic needle into a patient;
(d) moving the plunger proximally and distally over a distance less than the usable length of the syringe in order to check whether blood can be drawn into the syringe from the patient, at least a part of the said movement being permitted by the restrictor element, or part of it, rotating in the barrel;
(e) Administering the injection.

In a preferred form, the barrel has a cylindrical interior surface substantially free of discontinuities between proximal and distal ends of a full range of usable movement of the plunger head in the barrel.

A syringe barrel which is substantially free of discontinuities over its usable extent helps to make for smooth operation of the plunger, especially during the delivery stroke when an injection is being given to a patient. Any jolts in the operation of the syringe during the delivery stroke are normally felt by the patient, and it is desirable to avoid this happening. Discontinuities in the inner cylindrical surface which would not be engaged by the sealing face of the plunger head (piston) in normal use or in use during the delivery stroke are not considered to be in the “usable extent” of the barrel. For example, a reduced diameter portion of the barrel may be provided at the far proximal end of the usable extent of the barrel for preventing or hindering complete withdrawal of the plunger from the barrel: this would not be considered part of the “usable extent”.

Preferably, the syringe plunger is also substantially free of discontinuities for the majority of its length, e.g. greater than 70% of its length, preferably 80% and still more preferably 90% of its length. Standard syringe plungers are made in this way, and it is a considerable advantage of the present invention that in some embodiments it involves the use of a completely standard syringe with a standard barrel and plunger. Previous designs of auto-disable syringe use specially adapted plungers or specially adapted barrels in addition to locking components.

It is possible for a restrictor bobbin according to the invention to be retro-fitted to a standard syringe. Accordingly, in one aspect of the invention, an auto-disable syringe comprises a standard syringe having only a single unitary restrictor element or bobbin fitted therein to provide an auto-disable function. Preferably, unlimited repeated back and forth movement of the plunger of the auto-disable syringe is possible over a limited distance which is less than the full usable extent of the syringe, e.g. between 1 and 50% of the full usable extent, preferably between 1 and 25%, more preferably between 5 and 15%. By “standard syringe” is meant a disposable syringe which has no features whose primary purpose is to form part of an auto-disable system (i.e. a system to hinder or prevent more than one use of the syringe). The “standard syringe” barrel preferably has an undercut feature (reduced internal diameter) at or adjacent the proximal end.

The provision of lost motion or free relative movement of the plunger shaft over a limited predetermined extent allows repeated movement over a distance which is sufficiently small not to allow repeated injections with the syringe, or at least only to allow repeated injections of smaller quantities of fluid than the syringe would normally be used for or normally be capable of or suitable for delivering. This degree of movement may be sufficient to allow for aspiration/flash-back of blood for checking needle location in a patient. Alternatively or in addition, this degree of movement may be sufficient for repeated movements to assist reconstitution of powdered/lyophilised drug into solution.

It is preferable, though not essential, that the feature which allows aspiration of blood be operative at any position of the plunger so that the feature can be used whatever volume of injectable is contained in the syringe and also may be used to check the needle position in a patient prior to drawing a blood sample, i.e. when the syringe is substantially empty and the plunger in a distal position with respect to the barrel. Therefore, preferably the said limited repeated cycles of distal and proximal movement referred to above are permitted at substantially every relative position of the plunger and barrel over a the usable range. By “substantially every relative position of the plunger and barrel” is meant at least 80% of the extent of the range of relative movement of plunger and barrel.

The distance over which repeated movement is possible is, desirably, sufficient to aspirate a small volume of blood from a patient so as to check the position of the needle. All that is required for this function normally is that the aspirated blood be visible in the syringe. Factors which may need to be taken into account in determining the degree of movement required for this function may include, without limitation:
(a) the internal volume of the needle;
(b) if the needle is a separate entity, the internal volume of the syringe nozzle onto which the needle hub fits and any volume between the end of the nozzle and the internal base of the needle hub;
(c) any volume between the internal end of the syringe barrel and the plunger face when fully depressed;
(d) any “end float” of the plunger head: if the the plunger head is a separate entity from the shaft, then a certain amount of free play between the two is sometimes required to ensure the head “snaps on” to the shaft in manufacture;
(e) resilient deformation of the plunger head;
(f) the pressure drop which it is necessary to create to be sure that blood is aspirated;
(g) the volume of blood which needs to be present in the syringe barrel for the user to be able to discern its presence;
(h) a safety/error factor;
(i) the distance over which the user can discern movement easily: the “ergonomics” of the feature; and
(j) the diameter of the syringe barrel and plunger head.

The last of these points particularly will have a large effect on the volume as swept by the plunger head which corresponds to the distance over which the said repeated movement is possible.

The smallest possible volume for achieving this effect is about 10 microlitres: this might be the case e.g. if a short and thin (e.g. 1 cm, 30 gauge) needle is used which is moulded into the syringe barrel and if a plunger with an integrally moulded head is used, etc. In a 10 ml syringe this would correspond to 0.1% of the 10 ml swept volume of the syringe which equates to 0.1% of the usable length of the syringe. This is not precise since the usable volume of a syringe is often slightly more than its stated or graduated volume.

Normally a volume considerably greater than 10 microlitres would be required. For example, a large needle (e.g. 5 cm long, 18 gauge) may have an internal volume of approximately 50 microlitres. A standard luer nozzle has a dead space of about 50 microlitres and there will be additional dead space between the end of the nozzle and internal end of the needle hub. The plunger head of some syringes may have an end float of up to 1 mm which could correspond in a 10 or 20 ml syringe to 500 microlitres or more. In a 10 or 20 ml syringe a very small volume of blood may be more difficult to see in which case as much as 500 microlitres may be required. Adding these factors together with an allowance for error and for creating the pressure drop for withdrawing the blood might give a volume of as much as 2000 microlitres. In a 20 ml syringe this would correspond to 10% of the usable (graduated) swept volume of the syringe.

If it is desired that the syringe plunger should be capable of repeated movement of sufficient extent to agitate a drug powder and diluent mix, then it may be desirable to increase this range to as much as 50%. Of course, the larger the range of free movement, the greater the danger that this movement makes the syringe too easily re-usable for injecting drugs or other uses. The figure of 50% would probably be much too high for a 10 or 20 ml syringe, but for a very small syringe (0.5 ml or less) it may not be totally unreasonable from the point of view of preventing or at least hindering further use.

It can be seen that a large range of possibilities exist depending on the exact use to which the syringe is to be put. However, for most situations, a volume of between 50 and 500 microlitres would be preferable. 50 microlitres might be appropriate e.g. for a 5 ml syringe with a very small integral needle and where agitation of reconstituted drugs is not required. It would probably be desirable for this quantity to be more in the region of 100 or 150 or 200 microlitres, however, to allow a good margin for safety, human error, manufacturing tolerance, etc. 1000 microlitres might be appropriate for the same syringe where the ability to agitate reconstituted drugs is desirable, though this may still provide too great an opportunity for re-use of the syringe, and 500 microlitres may be more preferable.

Preferably, the distance over which the said repeated movement is possible is between 0.1% and 50% of the said barrel usable length, preferably between 1% and 25%. The lower end of this range might preferably be increased to 2%, 3% or 4% based on the example discussed above. The upper end of this range might preferably be reduced to 10% or 15% based on the example discussed above. However, these ranges should not be taken as limited to the particular syringe sizes discussed above which are presented by way of example only.

Accordingly, the said distance over which repeated movement is possible corresponds to a swept volume which is between 10 and 2,000 microlitres, preferably between 50 and 1000 microlitres, more preferably between 100 and 500 microlitres, or other absolute volume ranges corresponding to the percentage values of the total syringe volume mentioned above, for syringe total usable/measurable volumes of 0.5 ml, 1 ml, 1.5 ml, 2 ml, 2.5 ml, 3 ml, 5 ml, 10 ml and 20 ml.

The penultimate item in the above list of factors which may affect the desirable range of repeatable movement, factor (i), may require that a minimum distance is determined by what a user can practically work with. A reasonable range might be 0.5 mm to 20 mm, preferably 1 mm to 15 mm, more preferably 1.5 mm to 10 mm, still more preferably 2 mm to 7 mm, 2 mm to 5 mm, or about 3 mm or about 4 mm.

All the embodiments of the invention which are described herein are described principally with respect to a syringe having a nozzle, e.g. a luer connector, for attachment of a cannula, hypodermic or other needle or catheter line, etc. It will be appreciated that in every embodiment this nozzle could be replaced by a needle which is incorporated into the syringe at manufacture, e.g. moulded into the plastic of the barrel.

Alternatively, for any of the embodiments of the invention, the syringe could be provided not only with an integral needle but also with an integral needle shielding or retracting mechanism, a number of which are known in the art. All of these mechanisms are “active” rather than “passive”. Although some manufacturers describe their products as “passive”, they are not really “passive” in the true sense; that is to say they require a positive action by the user, even if this is only additional pressure on the plunger after administration of medication. Therefore, there is always a risk that the needlestick prevention mechanism will not be actuated by the user, allowing the syringe to be re-used; this is a particular risk in developing/emerging countries where syringe and needle re-use is common. A syringe with an auto-disable feature such as that of the present invention of course prevents further use of the syringe whether any needle retraction mechanism is actuated by the user or not. Therefore, there is no incentive for the user not to actuate the needlestick mechanism after using the syringe, since the syringe and needle cannot be re-used in any event. Thus, a syringe having an auto-disable mechanism as described herein, in combination with a known needlestick prevention mechanism, especially of the retracting needle variety, has distinct benefits over these two features when considered independently. For this reason, a further aspect of the present invention is a syringe according to any of the above aspects or incorporating a restrictor element according to any of the above aspects, where the syringe also incorporates an integral needle shielding or retracting mechanism. By “integral” is meant a needle and mechanism which are manufactured together with the syringe as opposed to a separate needle which is fitted to the syringe by the user.

It should also be understood that all of the following embodiments may be adapted to provide a frangible or weakened region on the plunger which is designed to break if excessive force is applied to the plunger. Alternatively the plunger may be made in more than one part which parts are designed to separate when excessive force is applied. In either case, the syringe is rendered inoperable or at least substantially inoperable.

Two embodiments of the invention will now be described, by way of example only and not in any limitative sense with reference to the accompanying drawings, in which:-

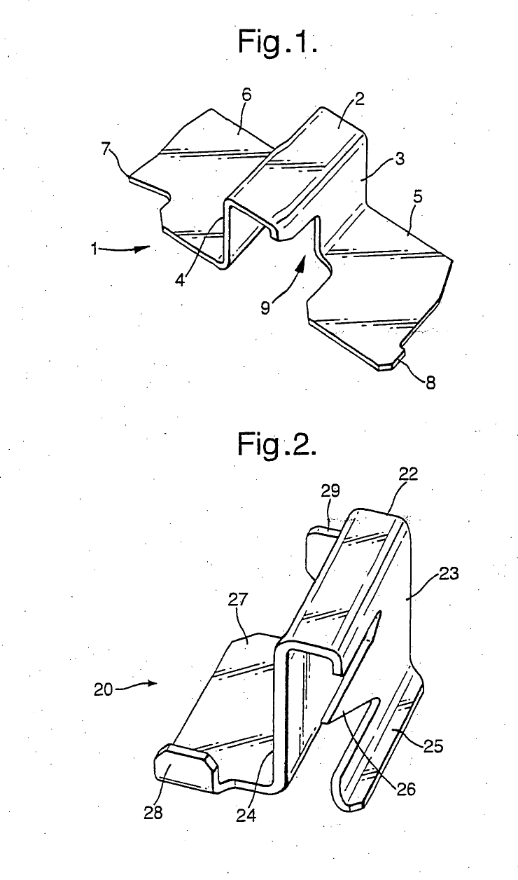

FIG. 1 is a perspective view of a barrel lock component of a first embodiment of the invention;

FIG. 2 is a perspective view of a plunger lock component of the first embodiment of the invention;

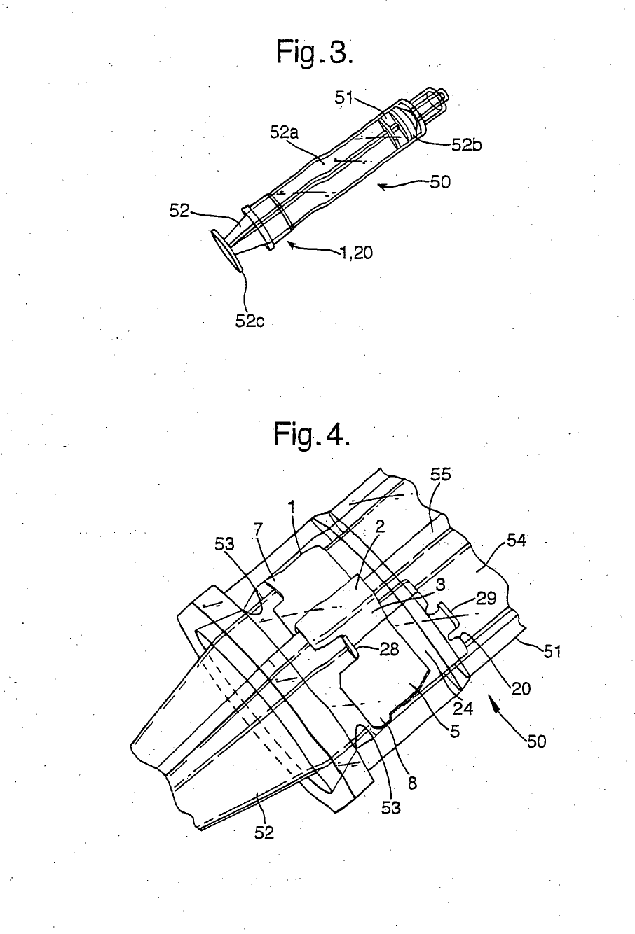

FIG. 3 is a perspective view of a syringe according to the first embodiment the invention, in its shipped state;

FIG. 4 is a partial view of the syringe of FIG. 3 in its shipped state, showing the positions of the barrel lock and plunger lock components;

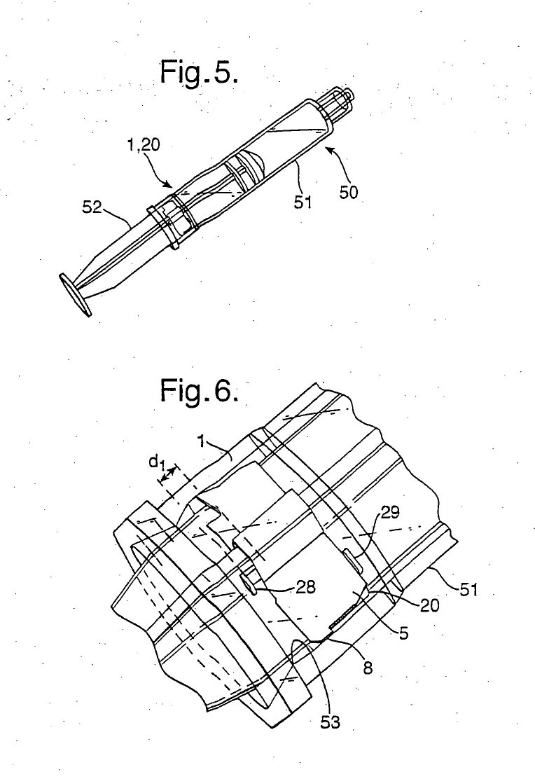

FIG. 5 is a perspective view of a the syringe of the first embodiment, with the plunger partially withdrawn;

FIG. 6 is a partial view of the syringe as shown in FIG. 5, showing the positions of the barrel lock and plunger lock components;

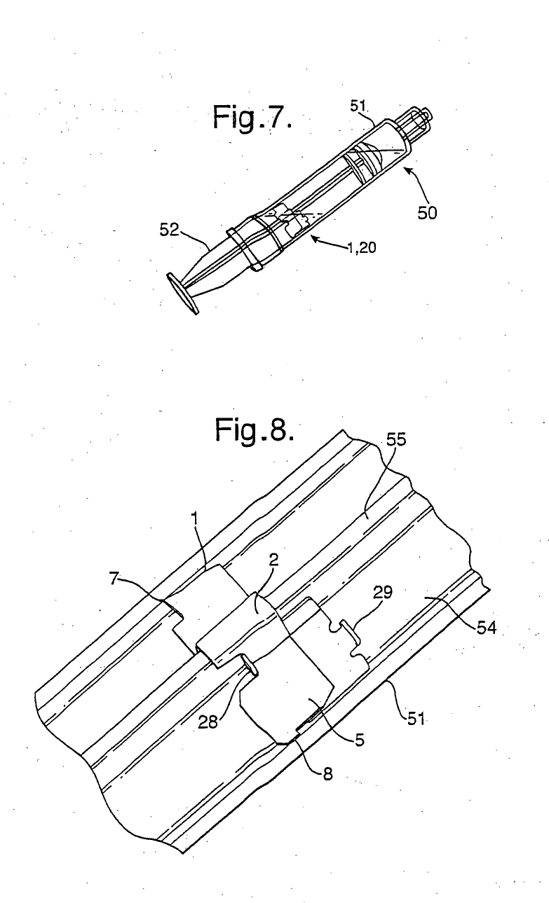

FIG. 7 is a perspective view of a the syringe of the first embodiment with the plunger partly depressed from the position shown in FIGS. 5 and 6;

FIG. 8 is a partial view of the syringe as shown in FIG. 7, showing the positions of the barrel lock and plunger lock components;

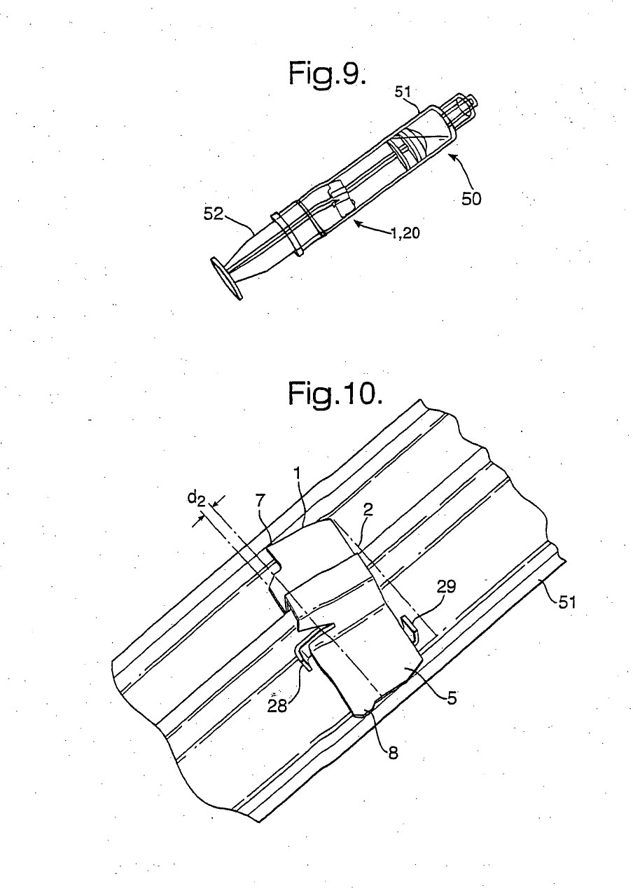

FIG. 9 is a perspective view of the syringe in a challenged state;

FIG. 10 is a partial view of the syringe as shown in FIG. 9, showing the positions of the barrel lock and plunger lock components;

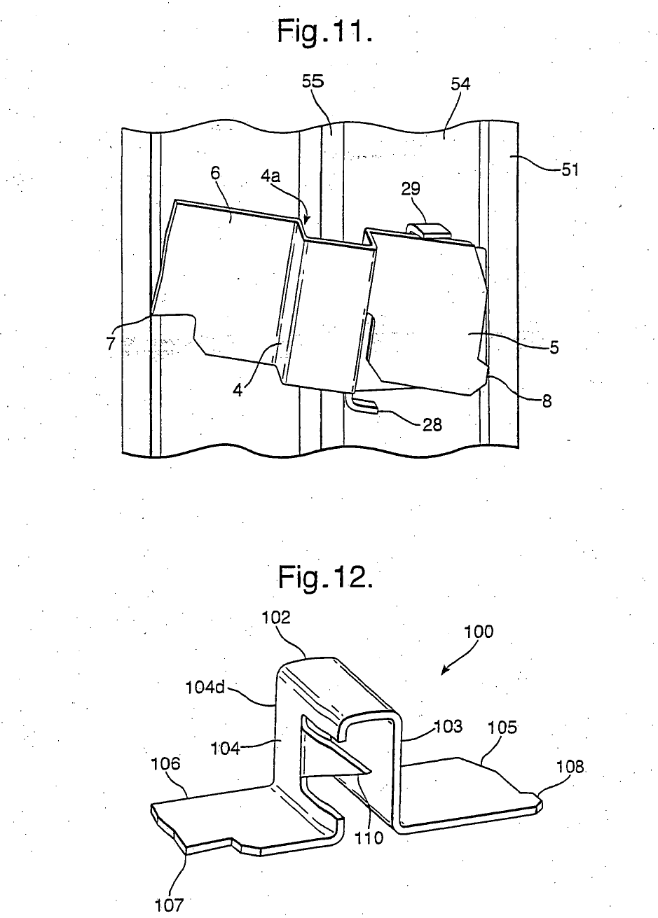

FIG. 11 is a side view of part of a syringe according to the first embodiment showing the barrel and plunger locks in a challenged state;

FIG. 12 is a perspective view of a single piece restrictor element of a second embodiment of the invention;

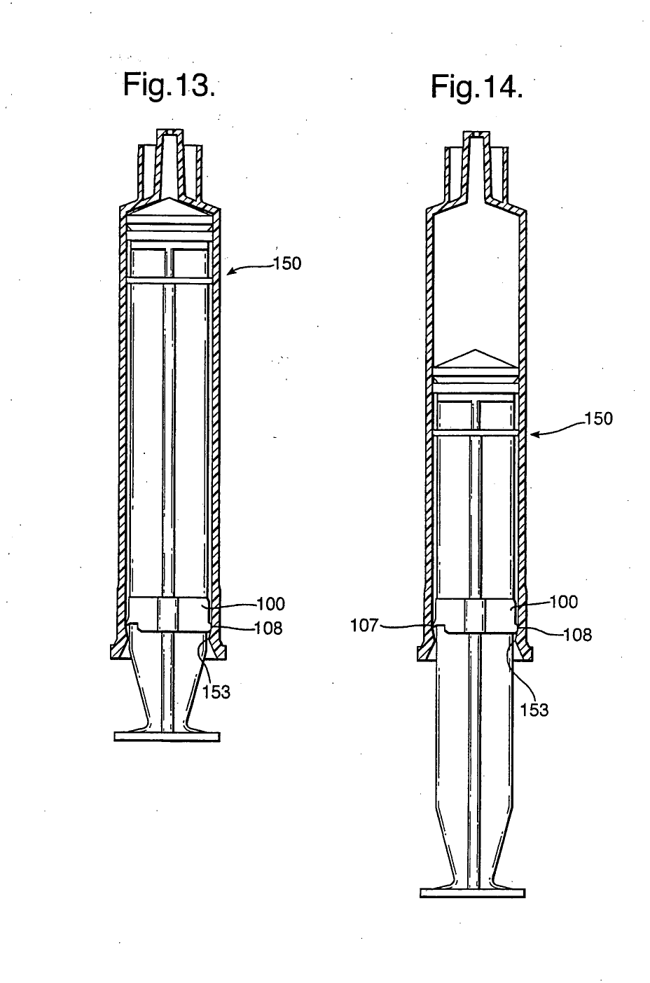

FIG. 13 is a longitudinal section of the syringe of the second embodiment in its shipped state;

FIG. 14 is a longitudinal section of the syringe of the second embodiment with the syringe plunger partly withdrawn;

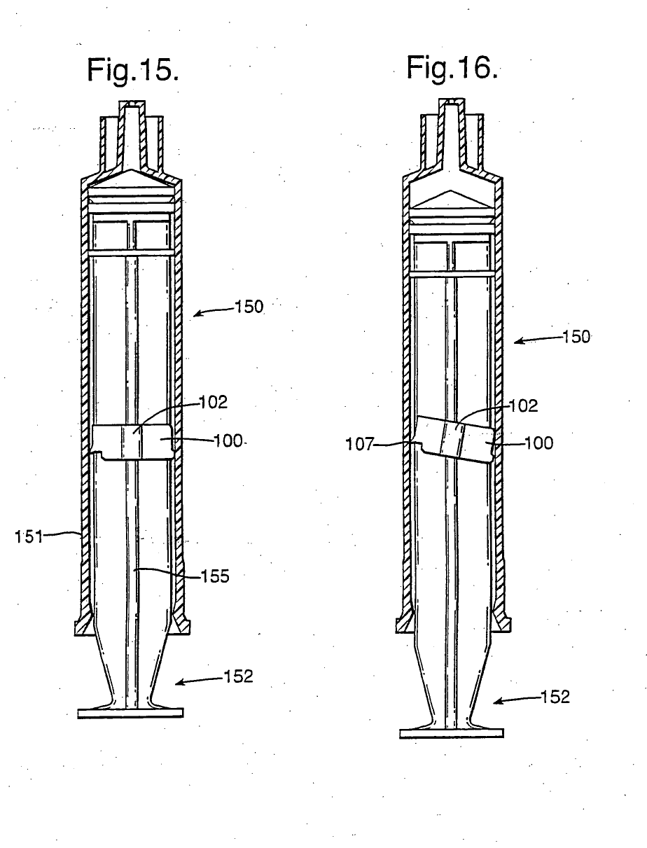

FIG. 15 is a longitudinal section of the syringe of the second embodiment with the syringe plunger re-seated after delivery of medication;

FIG. 16 is a longitudinal section of the syringe of the second embodiment showing the restrictor element in a challenged state;

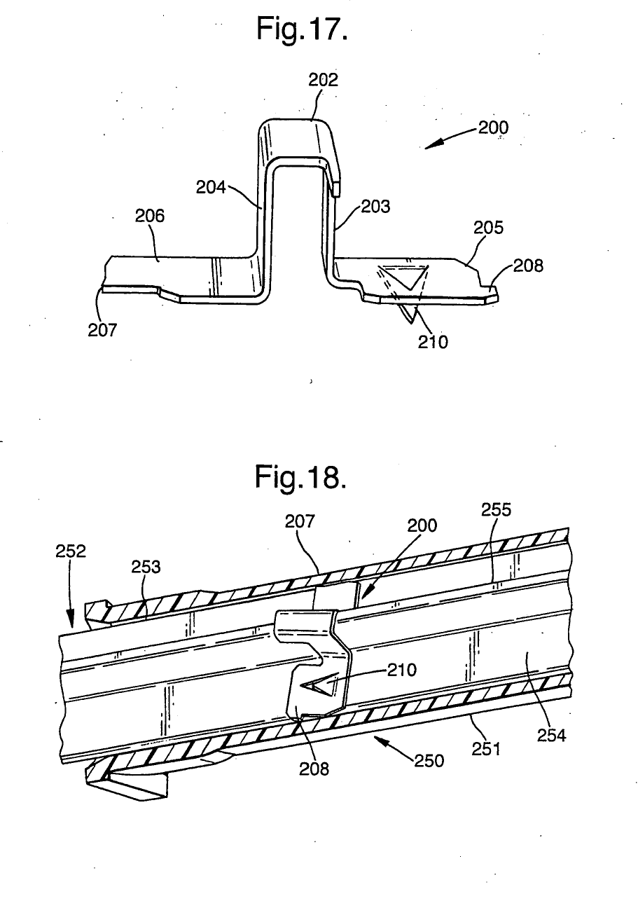

FIG. 17 is a perspective view of a modified version of the restrictor element of the second embodiment;

FIG. 18 is a perspective partial view of a syringe of the modified secondembodiment, showing the challenged state;

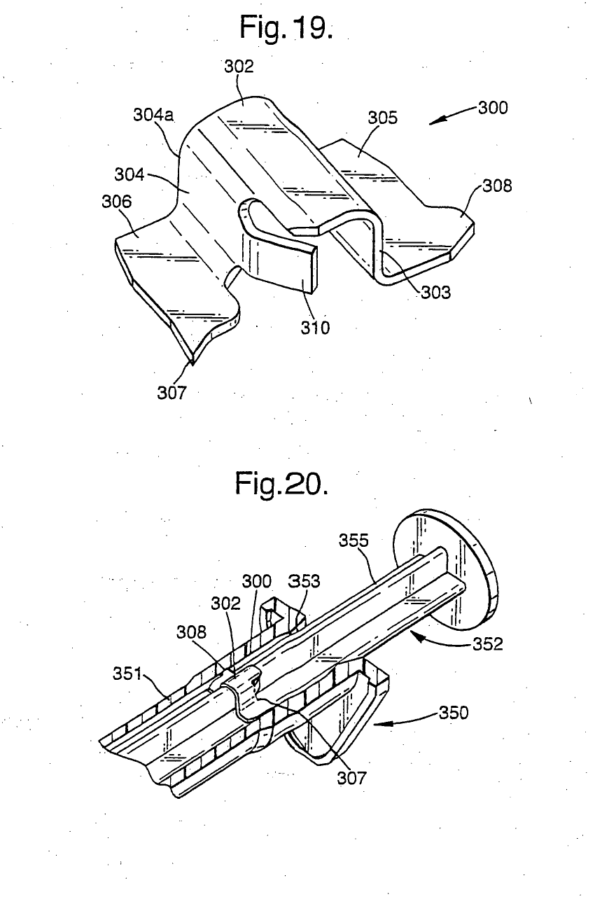

FIG. 19 is a perspective view of a single piece restrictor element of a third embodiment of the invention;

FIG. 20 is a perspective partial view of a syringe of the third embodiment;

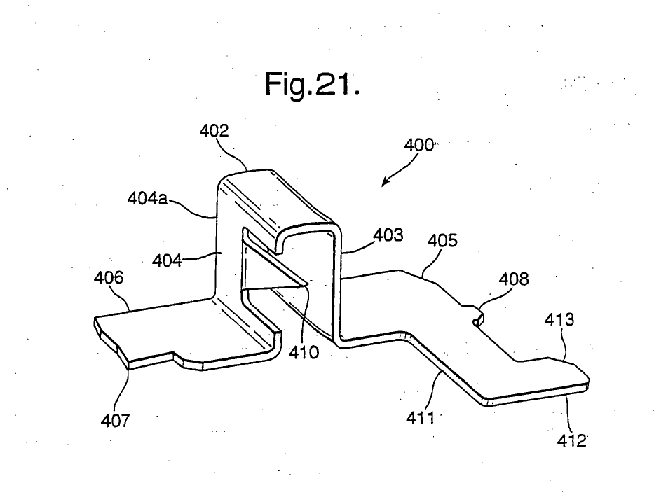

FIG. 21 is a perspective view of a single piece restrictor element of a fourth embodiment of the invention;

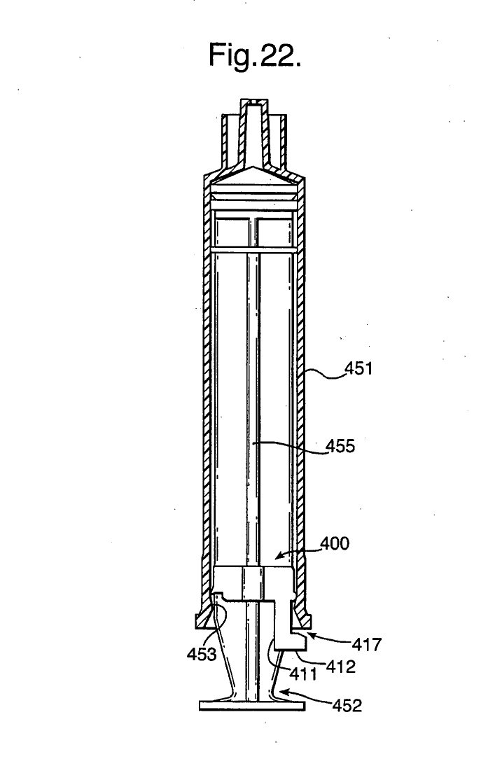

FIG. 22 is a longitudinal section of the syringe of the fourth embodiment in its shipped state;

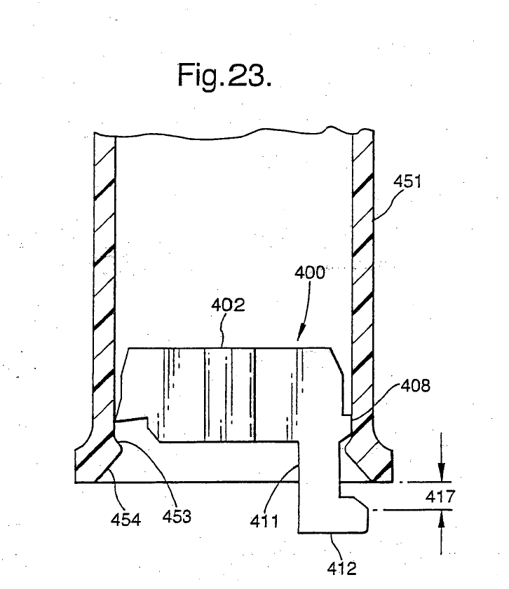

FIG. 23 is part of the sectional view of FIG. 22, on an enlarged scale;

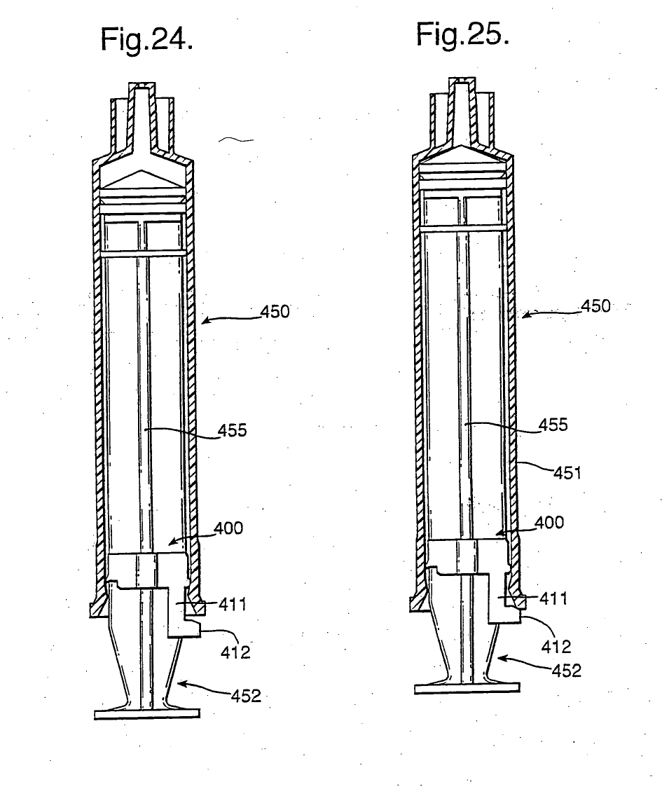

FIG. 24 is a longitudinal section of the syringe of the fourth embodiment with the plunger slightly retracted;

FIG. 25 is a longitudinal section of the syringe of the fourth embodiment with the plunger re-seated;

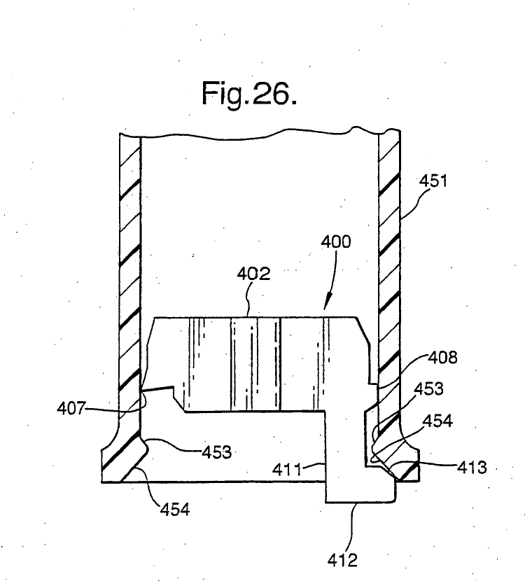

FIG. 26 is part of the sectional view of FIG. 25, on an enlarged scale;

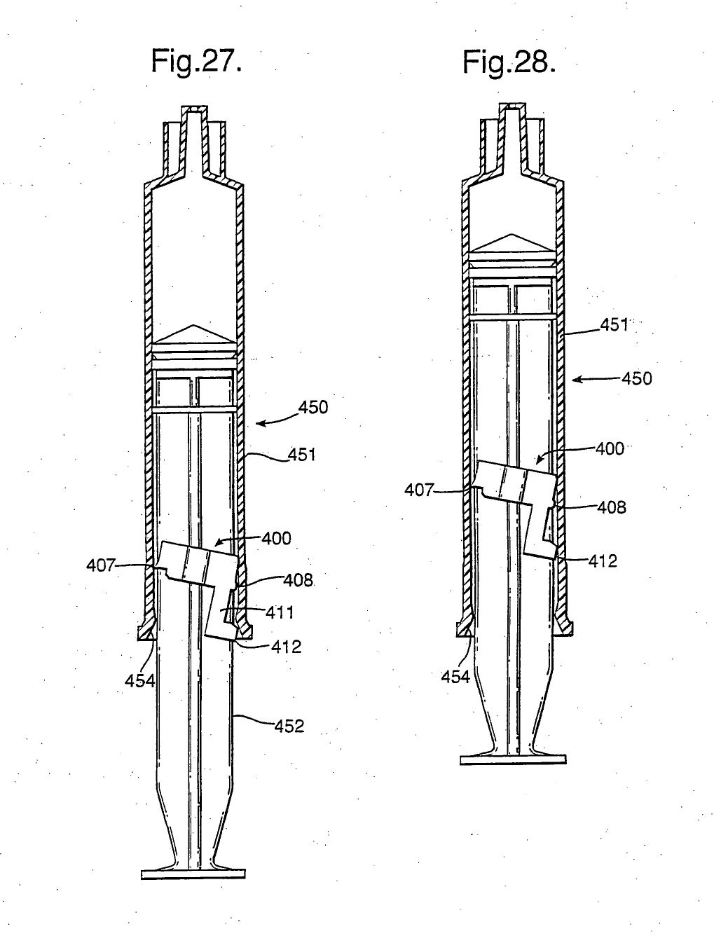

FIG. 27 is a longitudinal section of the syringe of the fourth embodiment showing the plunger being depressed from a substantially withdrawn position;

FIG. 28 is a longitudinal section of the syringe of the fourth embodiment in a challenged state;

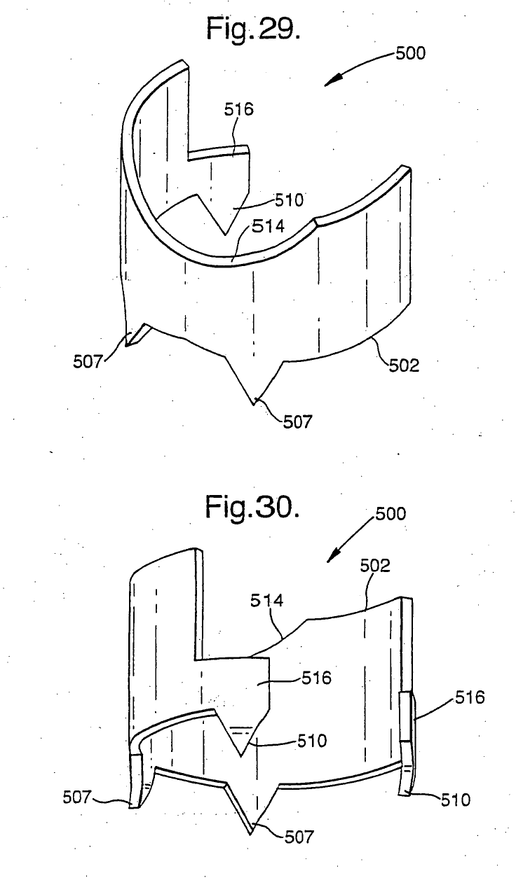

FIG. 29 is a perspective view from a first direction of a single piece restrictor element of a fifth embodiment of the invention;

FIG. 30 is a perspective view from a second direction of the restrictor element of FIG. 29;

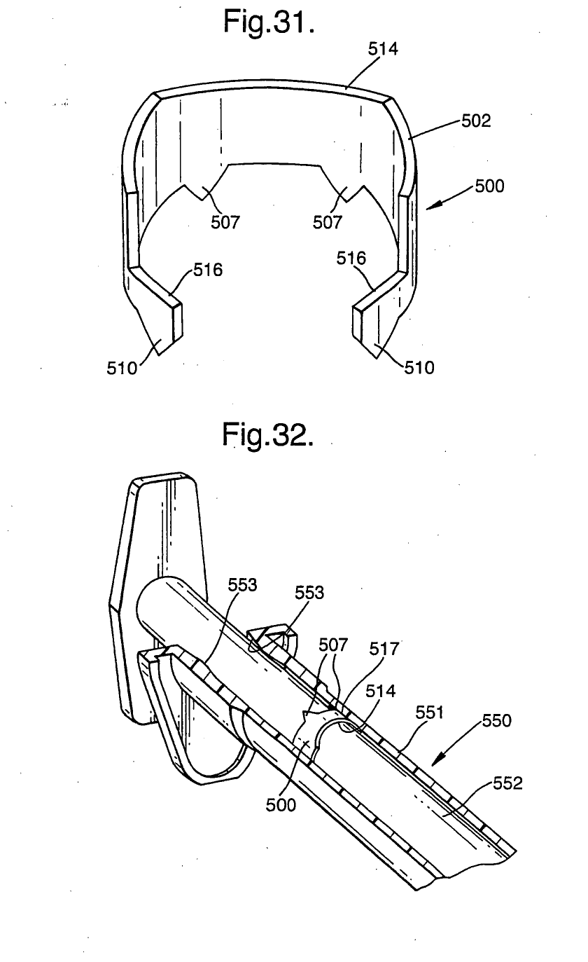

FIG. 31 is a perspective view from a third direction of the restrictor element of FIG. 29;

FIG. 32 is a perspective partial view of the syringe of the fifth embodiment;

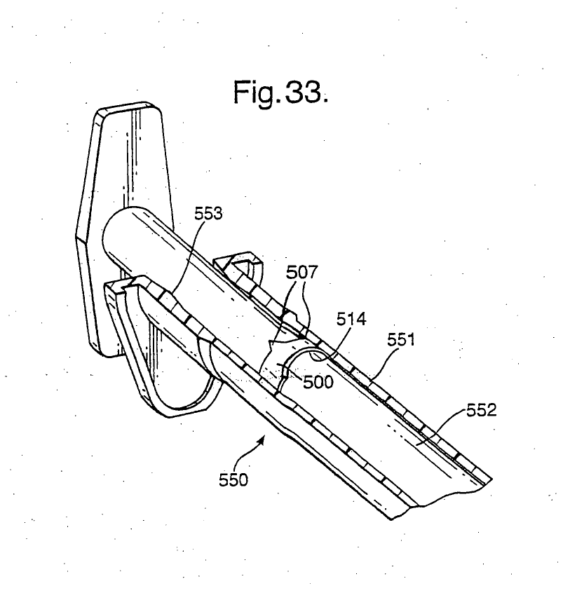

FIG. 33 is a perspective partial view of the syringe of FIG. 33, showing the restrictor element in a challenged state;

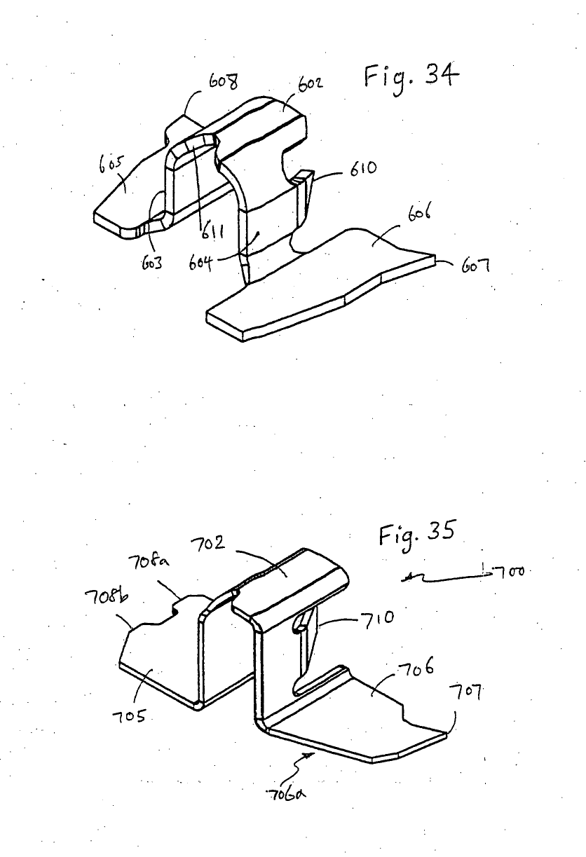

FIG. 34 is a perspective view of a sixth embodiment of restrictor element; and

FIG. 35 is a perspective view of a seventh embodiment of restrictor element.

The first embodiment is based on a standard 5 ml syringe with a cruciform section plunger shaft. Fitted between the plunger shaft and the barrel is a restrictor element. In this embodiment, the restrictor element comprises two pressed and formed stainless steel components 1, 20 termed the barrel lock and the plunger lock and shown, respectively, in FIGS. 1 and 2.

Referring to FIG. 1, the barrel lock 1 is shown. The terms “top” and “bottom” as used with reference to this embodiment refer to the top and bottom of the component as it is shown in FIG. 1. The terms “proximal” and “distal” will be used to refer to the left hand end and right hand end, respectively, of the component as it is shown in FIG. 1.

The barrel lock 1 is formed with a main arch 2 having a first side wall 3 and a second side wall 4. The stamping process which creates the barrel lock results in slightly rounded edges on one general face of the component, and somewhat sharper edges (a “burr”) on the opposed general face. Put another way, the edges on the top of the component and the edges on the bottom of the component will all have either rough or smooth edges respectively. For reasons which will be explained below, it is arranged for the sharper (or burred) edges to be generally on the bottom of the component, and hence, after forming of the stamped component, for the sharper edges of the main arch 2 to be on the inside if the arch.

Extending laterally from the bottom of each side wall 3, 4 are first and second plates 5, 6 respectively. The proximal outer tip of the second plate 6 is formed as a proximally oriented barb 7 which, when the component is installed in a syringe, bears against the inner wall of the syringe barrel to resist proximal movement of the barrel lock component 1 with respect to the syringe barrel. The outer edge of the first plate 5 has a protrusion 8 with rounded corners which is adapted, in use, to bear against the opposing side of the syringe barrel interior wall, to provide a reaction force for the barb 7. The first side wall 3 and first plate 5 are formed with a recess 9.

Referring to FIG. 2, the plunger lock 20 comprises a unitary body of pressed stainless steel formed into a main arch 22 with first and second side walls 23, 24.

The terms “top” and “bottom” as used with reference to this embodiment refer to the top and bottom of the component as it is shown in FIG. 2. The terms “proximal” and “distal” will be used to refer to the left hand end and right hand end, respectively, of the component as it is shown in FIG. 2. It should be noted, however, that the plunger component and barrel component are not both aligned in the same way when they are assembled together as a restrictor bobbin in a syringe.

The first side wall 23 of the main arch 22 is formed with an inwardly and proximally directed tang 26. This feature will be termed the distal barb, because it acts to prevent distal movement of the plunger with respect to the restrictor element (the restrictor element in this embodiment being constituted by the plunger lock of FIG. 2 and the barrel lock of FIG. 1). The bottom edge of the first side wall 23 is turned outwardly in a short flange 25.

Extending laterally from the bottom of the second side wall 24 is a plate 27, whose proximal end is formed as an upwardly turned flange, which will be called the proximal flange 28. At the distal end of the second side wall 24 is a further flange which will be called the distal flange 29. The function of these features will be explained later.

Turning now to FIG. 3, a syringe 50 is shown, with the components of FIGS. 1 and 2 installed, in its shipped state. Both the barrel lock 1 and plunger lock 20 are adjacent the extreme proximal end of the syringe 50. The proximal end of the syringe 50 together with the barrel lock 1 and plunger lock 20, are shown in more detail in FIG. 4.

The syringe comprises a barrel 51 and plunger 52. The barrel 51 includes an undercut 53 of reduced internal diameter at the proximal end of the barrel 51. The undercut 53 is a standard feature of most syringes; its purpose is to prevent accidental removal of the plunger from the barrel. The plunger assembly 52 comprises a plunger shaft 52a of generally cruciform section, that is to say in transverse section will have the shape of a cross with four flanges at 90 degrees to each other. The plunger shaft is fitted with an elastomeric plunger or plunger head 52b at its distal end. At its proximal end, the plunger shaft terminates in a disc shaped plate 52c for a user to manipulate the plunger assembly 52. All these components are conventional.

The barrel lock 1 and plunger lock 20 are each installed with their respective main arches 2, 22 located on respective flanges 55, 54 of the plunger at 90 degrees to each other. The barrel lock 1 is installed so that first side wall 3 and plate 5 extend over the plunger lock 20. The main arch 2 of the barrel lock has an internal width which is somewhat greater than the combined thickness of the plunger flange 55 and the plate 27 of the plunger lock, on which the barrel lock sits (see FIG. 4). This allows for a degree of rotation of the barrel lock 1 with respect to the plunger 52. The barb 7 of the barrel lock 1 touches the internal wall of the barrel on one side whilst the protrusion 8 from the barrel lock touches the opposing side of the interior wall of the barrel. The plunger lock 20 is arranged so that its second wall 24 is located between the plunger flange 54 and the first plate 5 of the barrel lock 1. The first wall 23 (not visible in FIGS. 3 and 4) of the plunger lock includes the distal barb 26 which touches the side of the plunger flange 54 on its side which is unseen in FIGS. 3 and 4. The distal barb 26 of the plunger lock is biased towards the flange 54of the plunger shaft by virtue of the geometry of the main arch 22 and the resilience of the stainless steel of which it is made. The proximal flange 28 of the plunger lock is received in the recess 9 in the barrel lock 1.

There are essentially four normal modes of operation of the syringe: injection of medicament using a hypodermic needle, administration of medicament into an i.v. line, withdrawal of blood using a hypodermic needle and withdrawal of blood via an i.v. line.

When injecting liquid medicament using a hypodermic needle, the sequence of steps is normally as follows. Firstly, a hypodermic needle is fitted to the syringe, if it does not already have an integral needle. The next step is to insert the needle into a vial of medicament and draw back the plunger to draw up the liquid medicament. As the plunger is drawn back (i.e. moved in the proximal direction), the shaft moves freely past the barrel lock 1. The distal barb 26 of the plunger lock is biased against the plunger shaft, but the barb 26 is oriented so as to create only a small resistance to proximal movement of the plunger. The plunger lock 20 moves proximally a short distance before the distal flange 29 of the plunger lock engages a proximal edge of the first plate 5 of the barrel lock 1. This position is shown in FIG. 6.

At this point, because the distal barb 26 produces a small degree of resistance against proximal movement of the plunger, further proximal movement of the plunger 52 will cause the distal flange 29 of the plunger lock 20 to bear against the first plate 5 of the barrel lock and thus tend to rotate the barrel lock. As discussed above, limited rotation of the barrel lock is in general possible because there is a certain amount of free play between the main arch 2 of the barrel lock and the plunger flange 55 on which it is located. However, in the position shown in FIGS. 5 and 6, such rotation is substantially inhibited by the protrusion 8 on the outer edge of the first plate 5 butting against the undercut 53 at the proximal end of the syringe barrel 51.

Further proximal movement of the plunger therefore does not result in substantial movement of the barrel and plunger lock components in any sense. FIG. 5 shows the syringe with the plunger withdrawn approximately half way along the length of the barrel: the barrel lock and plunger lock components remain in the same position, as shown in FIG. 6.

Normally, when drawing up medicament, more will be drawn up than is actually needed. It is also normal to have a small amount of air in the syringe as well. The next step, therefore, is normally to move the plunger distally to expel any air and any excess medication. The distal barb 26 of the plunger lock 20 is oriented such as to prevent distal movement of the plunger with respect to the plunger lock 20, so distal movement of the plunger will carry the plunger lock 20 with it. For a small distance d1 as shown in FIG. 6, the plunger lock 20 will move relative to the barrel lock 1; the two components are freely slidable with respect to each other over this small distance. In this particular embodiment, in which the syringe has a total usable volume of 5 ml, this distance is 2 mm (corresponding to a volume of about 0.2 ml). Movement of the plunger beyond this distance will cause the barrel lock 1 to be carried distally with the plunger lock along the barrel. Normally, the step of expelling air and excess medicament will involve more than 2 mm of distal movement of the plunger, such that the barrel lock 1 will be carried along down the syringe barrel with the plunger lock and plunger. This configuration-is shown in FIGS. 7 and 8.

It may be that the degree of movement required to carry out the step of expelling air and medicament is smaller than the distance d1, in which case the barrel lock 1 will not be moved. It is also possible that the air/medicament expulsion step may not carried out at all in some circumstances. The overall operation of the syringe and its auto-disable features remains essentially unchanged in this event, as will be apparent from the following description.

The next step is insertion of the hypodermic needle into the patient, followed either by delivery of the fluid or by an aspiration procedure. An aspiration procedure is used if it is desired to determine whether the needle tip is located in a blood vessel or not. The plunger is cycled back and forth one or more times: if blood appears in the barrel, this means the needle tip is located in a blood vessel and, conversely, if no blood appears then the conclusion is that the needle tip is in muscle. Depending on whether the syringe user wishes to inject into muscle or into a blood vessel, the position of the needle may be changed as necessary and the aspiration procedure repeated. Therefore, in order to allow for aspiration, it must be possible to move the plunger proximally to a limited extent from the position shown in FIGS. 7 and 8, then return it to that position and then be able to repeat this cycle of movement indefinitely.

Assuming that an air and excess medicament expulsion step has been performed, which would be normal, the syringe plunger will be at a point along the syringe barrel corresponding to the volume of medicament desired by the user; the barrel lock 1 will no longer be butted against the undercut 53 and the plunger lock 20 will be at the distal end of its range d1 of free play relative to the barrel lock 1. This is the position shown in FIGS. 7 and 8.

Initially, as the user draws the plunger back to perform an aspiration procedure, the plunger lock 20 will move back with it due to the interaction of the plunger lock barb 26 with the plunger. The barrel lock 1 will remain in the position shown in FIGS. 7 and 8 until the plunger has been moved through a distance corresponding to the free play distance d1 (see FIG. 6). At this point, the distal flange 29 of the plunger lock engages with the barrel lock 1 and biases the barrel lock in the proximal direction. The barrel lock barb 7 is oriented so that its engagement with the interior surface of the syringe barrel resists proximal movement of the barrel lock. The opposite side of the barrel lock is not prevented from moving proximally, however, since the projection 8, with its rounded corners, is not designed to resist movement along the barrel wall and, in the FIG. 7/8 position, is not butted against the syringe barrel undercut 53. The barrel lock 1 therefore tends to rotate about the tip of the barb 7, allowing the plunger lock 20, and the plunger, to be moved further in the proximal direction.

After the barrel lock 1 has been rotated to a certain extent, it will wedge in the barrel, thereby preventing further proximal movement of the barrel lock and also the plunger lock. This wedging of the barrel lock will occur at any point along the length of the barrel provided the barrel lock is not adjacent the undercut 53. FIGS. 9 and 10 show this configuration.

As previously discussed, the main arch 2 of the barrel lock 1 has an interior width somewhat greater than the thickness of the plunger flange 55 on which it sits; therefore the rotating/camming action of the barrel lock is not resisted initially by the interengagement of the barrel lock and plunger flange 55 on which it sits. As the barrel lock continues to rotate, the inner distal edge 4a of the second wall 4 of the barrel lock engages the plunger flange 55. This is best seen in FIG. 11. The barrel lock is dimensioned so that the edge 4a engages the plunger flange 55 at approximately the same position in which the barrel lock as a whole wedges across the syringe barrel. At this point, if further force is applied to the plunger in the proximal direction, the distal edge 4a of the second wall 4 engages with the plunger flange 55 in the manner of a barb, so as to resist further proximal movement of the plunger. As discussed above, the barrel lock component is of stamped stainless steel plate, which has been stamped in a direction such as to cause (after forming) the inner side of the edges of the arch 2, including the inner edge 4a, to be somewhat sharp. As the user moves the plunger in a distal direction from the position shown in FIGS. 9 to 11, the plunger lock will move distally with respect to the barrel lock and the barrel lock will rotate about the tip of its barb 7 until the plunger and restrictor element have returned to the configuration shown in FIGS. 7 and 8. At this point, the user may either deliver the medication by depressing the plunger distally or may if necessary repeat the aspiration cycle.

It will be appreciated that the rotating/camming of the barrel lock 1 provides additional free movement of the plunger, shown as distance d2 in FIG. 10. The total free play of the plunger in the barrel is therefore d1+d2. The extent d1 of the repeatable movement of the plunger without rotation of the barrel lock 1 is in this embodiment 2 mm. The extent of free play d2 provided by rotation/camming of the barrel lock is in this embodiment also about 2 mm, though this is less precise because it will depend to some extent on the force applied to the plunger in a proximal direction.

The dimensions of the barrel lock and plunger lock are obviously easily changed to alter d1. More subtly, the overall width of the barrel lock, the internal width of the main arch, the axial lenth of the main arch, the length of the barb 7, etc will affect the degree of free play d2 due to rotation of the barrel lock. These dimensions will also change depending on the exact dimensions of the syringe—internal barrel diameter, thickness of plunger flanges, etc. In this embodiment, the width of a plunger flange is approximately 0.04″ and the internal diameter of the barrel is approximately 0.47″ along most of the length of the barrel and approximately 0.45″ at the undercut. The skilled person will of course understand that these dimensions may be changed to accommodate all different sizes of syringe and to allow selection of the desired amount of free play d2, by careful analysis of the geometry and/or by trial and error. The barrel lock and plunger lock in this embodiment are made from 0.010″ thickness stainless steel. The axial length of the main arch of the barrel lock and plunger lock is approximately 0.2″. The overall span of the barrel lock is 0.48″ of which the barb 7 makes up 0.10″. The internal width of the main arch of the barrel lock is approximately 0.07″, whilst that of the plunger lock is 0.044″. The restrictor bobbin assembly, comprising barrel lock 1 and plunger lock 20, is designed so that the aspiration operation may be carried out repeatedly without actuating the auto-disable function of the restrictor bobbin, whatever the position of the plunger. However, the functioning of the restrictor is slightly different if the aspiration procedure is carried out when the barrel lock 1 is still butted against, or is adjacent to, the barrel undercut 53. In this event, there is of course no limit on the extent of proximal movement of the plunger since the barrel lock will not rotate, or will not rotate sufficiently to cause it to wedge against the barrel and plunger. The auto-disable function of the syringe is activated as soon as the barrel lock is moved in a distal direction sufficiently far from the undercut 53 that it is free to rotate to its fully wedged position without encountering the undercut. Then, if the user moves the plunger again proximally, the barrel lock will rotate and wedge against the barrel and plunger.

The aspiration step complete, the medicament is then administered by depressing the plunger completely, i.e. moving it as far as possible in a distal direction. This will result in the plunger lock 20 moving distally with respect to the barrel 51. The plunger lock will of course carry the barrel lock with it, as the flange 28 engages in the recess 9. This is the configuration shown in FIGS. 7 and 8. i.

When the plunger is fully depressed, the syringe is then disabled against subsequent substantial movement of the plunger in a proximal direction, as described above.

Applying a substantial force to the plunger in a proximal direction may cause the barrel lock barb 7 to penetrate deeply into the plastic of the syringe barrel and/or the edge 4a to penetrate the plunger flange 55. This of course acts to increase the ability of the restrictor to prevent withdrawal of the plunger. Although, as the barb 7 and/or edge 4a penetrates the plastic, it may be possible to withdraw the plunger very slightly further in a proximal direction, this degree of movement is not significant. The wedging/camming action therefore provides a very secure way of locking the syringe.

Application of extreme force may result in the barrel lock being dragged along the syringe; however, in this case, the interior of the barrel will be severely damaged, thus rendering the syringe unusable. In this embodiment, the barb 7 is in fact dimensioned such that it will not penetrate completely through the wall of the barrel. However, in a modified version of this embodiment, the barb 7 is slightly longer and is designed so that, if extreme force is applied, it penetrates the barrel wall completely and cuts completely through the wall as it is dragged in the proximal direction.

As mentioned above, the other principal possible uses for a syringe are withdrawal of blood using a hypodermic needle, or delivery of medication via an i.v. line or withdrawal of blood through an i.v. line.

Blood withdrawal using a hypodermic needle involves a slightly different sequence of steps than delivery of medication. The first step is to fit a needle to the syringe (if there is no integral needle) with the plunger in the fully seated “as shipped” position. The user then inserts the needle into the patient and attempts to locate the needle tip in a vein. Often this will require more than one attempt. Starting from the position shown in FIGS. 3 and 4, it is possible to draw back the plunger as far as desired to attempt to withdraw blood. If unsuccessful, the plunger is re-seated (moved distally) before repositioning the needle in the patient and making another attempt to withdraw blood. If distance of the initial proximal movement was d1 or less, then re-seating the plunger will simply return the barrel and plunger locks to their original FIGS. 3/4 configuration. If the initial proximal movement was more than d1, then subsequent re-seating of the plunger will move the barrel lock away from the undercut 53 in the syringe barrel, which will prevent further proximal movement of the plunger and thus prevent withdrawal of any blood sample. Therefore withdrawal of blood samples is quite possible with this design, but care is needed to use only the d1 distance when aspirating to check for the location of the needle tip in the patient.

One of the many advantages of this design is the fact that it is based around a standard syringe, which requires only the addition of two small stamped components to turn it into an auto-disable syringe. The steel components are readily adaptable to suit any size of syringe, and the componts may easily be retrofitted to standard syringes which have already been manufactured. Of course it is equally possible to incorporate assembly of the barrel and plunger locks into the overall syringe assembly process.

To assemble the barrel, plunger and lock components together, two approaches are possible. In the first approach, the components are mounted on the plunger in the position which they will occupy in the final product as shipped. First the plunger lock is mounted on one flange of the plunger; the proximal direction barb 26 resiliently bears on the plunger flange, keeping the component securely in place. The barrel lock is then fitted to a flange at 90 degrees to the first one; since the barrel lock is a loose fit, it is ensured that the plunger flange on which the barrel lock sits is oriented upwardly.

The plunger is then inserted into the barrel. When the plunger has been inserted sufficiently far that the lock components encounter the syringe undercut 53, a moderate additional force is applied to the plunger to move the barrel lock past the undercut. The resilient polypropylene plastic of the syringe barrel allows the locks to pass without causing any permanent deformation. Conveniently, the plunger lock proximal barb 26 prevents the lock components from sliding along the plunger, and the lock components are thereby kept at the correct point on the plunger as they are inserted into the barrel. It is quite easy to do this assembly process by hand. In an automated process the same technique could be used, though for additional security it may be preferable to have a tool engage directly with the lock components (e.g. at flange 29) as the plunger is being fitted to the barrel.

The second approach is to assemble the barrel and plunger and then to mount the lock components on the protruding part of the plunger as described above, apply a tool e.g. to the flange 29 of the plunger lock and then slide the components along the plunger, forcing them past the barrel undercut and into the correct position.

A second embodiment of the invention is shown in FIGS. 12 to 16. FIG. 12 shows a component, which will be called the restrictor bobbin 100, which is similar in most respects to the barrel lock 1 of the first embodiment. As with the first embodiment, the bobbin 100 is intended for use in a standard 5 ml syringe. The main differences from the first embodiment are that a barb is formed in the equivalent of the second side wall 4 of the barrel lock 1 of the first embodiment, and the recess 9 of the barrel lock 1 is omitted.

When describing the second embodiment, the terms “top”, “bottom”, “proximal” and “distal” have the same general sense as in the first embodiment. Thus, the restrictor bobbin 100 is formed with a main arch 102 having a first side wall 103 and a second side wall 104. It is made from stamped stainless steel plate of thinckess 0.010″, with the sharper edge resulting from the stamping being on the bottom general face and on the inside of the main arch 102.

Extending laterally from the bottom of each side wall 103, 104 are first and second plates 105, 106 respectively. The proximal outer tip of the second plate 106 is formed as a proximally oriented barb 107 which, when the component is installed in a syringe, bears against the inner wall of the syringe barrel to resist proximal movement of the restrictor component 100 with respect to the syringe barrel. The outer edge of the first plate 105 has a protrusion 108 with rounded corners which is adapted, in use, to bear against the opposing side of the syringe barrel interior wall, to provide a reaction force for the barb 107. The second side wall 104 is formed with a stamped out proximal plunger barb 110 which in use is designed to bear resiliently against the plunger of a syringe in which the restrictor 100 is installed.

FIGS. 13 to 16 show the second embodiment in full, with the bobbin 100 of FIG. 12 installed in a 5 ml syringe 150 of standard design.

The operation of the second embodiment is in most ways very similar to that of the first embodiment. The restrictor bobbin 100 starts out in the shipped syringe 150 (FIG. 13) in the same position as that of the barrel lock 1 of the first embodiment as shown in FIGS. 3 and 4. Initial proximal movement of the plunger to draw up medicament or other fluid is not inhibited by the restrictor bobbin since its projection 108 is butted against the syringe barrel undercut 153 to prevent rotation/camming of the bobbin 100. FIG. 14 shows the position of the plunger after drawing up a volume of medicament somewhat less that the maximum capacity of the syringe. The bobbin 100 has not moved either axially or rotationally; the interaction of the projection 108 and barrel undercut 153 prevent proximal movement of the right side of the bobbin (as seen in FIG. 14) whilst the engagement of the barb 107 with the interior surface of the barrel 151 prevents proximal movement of the left side of the bobbin (as seen in FIG. 14).

Distal movement of the plunger 152 to expel air and/or excess medicament will carry the bobbin 100 with it due to engagement of the plunger barb 110 with the plunger. The plunger barb 110 is not shown in FIGS. 13 to 16 but may be seen in FIG. 12; it may be imagined that the barb 110 will be in engagement with the plunger flange/wing 155 over which the bobbin main arch 102 is mounted, and the direction of the barb 110 is such as to inhibit relative distal movement of the plunger with respect to the bobbin 100. Once the restrictor bobbin has moved a small distance in a distal direction from its start position, the restrictor bobbin is no longer hindered by the barrel undercut 153 from rotating; therefore in the position shown in FIG. 15, the auto-disable and unlimited free play features of this design become activated.

The next step in the operation sequence is to insert the needle into a patient; the aspiration step is then performed as with the first embodiment. Proximal movement of the plunger of up to approximately 4 mm (corresponding to about 0.4 ml) is possible due to rotation of the bobbin 100 about the barb 107. This will be explained in more detail below in relation to FIG. 16. Once the aspiration step has been completed and the user is satisfied that the needle is in the correct position in the patient, the medication is administered by depressing the plunger completely. This position is shown in FIG. 15. The syringe is now disabled.

FIG. 16 shows the bobbin 100 being challenged as a user attempts to withdraw the plunger 152. This proximal movement of the plunger will tend to rotate the bobbin as the barb 110 experiences a small force in the proximal direction due to its contact with the plunger. As with the first emobodiment, the barb 107 resists proximal movement with respect to the barrel, causing the bobbin 100 to rotate as the plunger is pulled back. As the bobbin 100 rotates, it wedges in the barrel and the leading edge 4a of the second wall of the main arch 102 engages with the plunger flange 155 to prevent proximal movement of the plunger with respect to the bobbin 100. The dimensions of the bobbin—its overall width, the size of the barb 107 and the projection 108, the internal width of the main arch 102, all affect the way it functions and will, depending on the relevant dimensions of the syringe which it is designed to fit, determine the degree of free play which is provided. Although the dimensions of the bobbin are important, they are relatively easily established by one of ordinary skill in the art by analysis of the geometry and/or by trial and error.

As with the first embodiment, the barb 107 is dimensioned so that it does not protrude right through the barrel wall, though in a first modification of the second embodiment it could do so.

The disabling function of the bobbin will operate at substantially any position of the plunger in the barrel, the exception of course being when the bobbin is adjacent the syringe undercut 153. For this reason, the free play permitted by rotation of the barrel lock provides for the ability to aspirate at substantially any position of the plunger, as with the first embodiment. As with the first embodiment, distal movement of the plunger 152 will cause the restrictor bobbin 100 to rotate in the opposite sense and to “un-wedge”. It will generally pivot about the barb 107 over, in this example, a distance of about 4 mm. Further distal plunger movement beyond this (requiring considerable force) will cause the restrictor to be dragged along the barrel by the plunger, thereby damaging the barrel and rendering the syringe substantially unusable.

In this second embodiment, therefore, the “lost motion” is provided entirely by the rotating/camming action of the restrictor element. The extent of lost motion created by this action may be predetermined by making minor adjustments to the dimensions of the restrictor—its overall width from barb 107 to projection 108, the internal width of the main arch 102, the dimensions of the barb 107, etc. The exact degree of lost motion will depend to some extent on how hard the plunger is pulled proximally, as discussed above in relation to the first embodiment.

The second embodiment may not be suitable for withdrawing blood samples. This is because, in the second embodiment, all free play is provided by rotation of the bobbin 100 rather than by relative movement of a barrel and plunger lock, as in the first embodiment. For this reason, if a user attempts to apirate blood, fails to do so, and then re-seats the plunger, the bobbin will almost inevitably have moved far enough away from the barrel undercut that it is it freed to rotate, which of course means that further substantial proximal movement of the plunger is prevented.

A modified form of the second embodiment is shown in FIGS. 17 and 18. This embodiment works in almost exactly the same way as the second embodiment and the operation will not be described in detail. Essentially the only difference with this modified version of the second embodiment is that the plunger barb 110 of the second embodiment is moved to a different position on the bobbin, and becomes plunger barb 210 in FIG. 17. FIG. 17 shows the detail of the bobbin 200 of the modified second embodiment in a view similar to FIGS. 1 and 12, whilst FIG. 18 shows the bobbin 200 in a challenged state in a syringe 250. Whilst this arrangement works well, the unmodified version on the second embodiment is preferred because the forces on the plunger when it is moved distally are applied more centrally, making the action of depressing the plunger smoother.

Assembly of the second embodiment is very simple. The technique is essentially the same as for the first embodiment except of course that only one lock component needs to placed onto the plunger. The proximal barb 110, 210 serves the same function during the assembly process as the proximal barb 26 of the plunger lock of the first embodiment: that is to say, it prevents movement of the bobbin as the plunger is inserted into the barrel. Furthermore, in the unmodified second embodiment, the proximal barb 110 acts to retain the bobbin 100 on the plunger.

The first and second embodiments, and the modified second embodiment, relate to standard 5 ml syringes such as those produced by B. Braun Melsungen AG under the trade mark Omnifix®. However, the restrictor bobbin components may be used in other 5 ml syringes with minimal or no changes to the dimensions. The bobbin components may also be adapted for use in standard syringes of other sizes, e.g. 1 ml, 2 ml, 3 ml, 10 ml, 20 ml, 30 ml or 50 ml (or indeed any size), by altering the dimensions of the components. All that is required is that the syringe have a cruciform plunger shaft, that an undercut be formed in the barrel at or near the proximal end and that there is sufficient clearance between the plunger shaft and the interior barrel wall for the restrictor bobbin component or components to fit between the barrel and plunger. Of course, if necessary, an undercut feature could be added as a separate component to a syringe which is not already equipped with such a feature. The third embodiment described below relates to a standard 1 ml syringe as produced under the trade mark Omnifix® by B. Braun Melsungen AG.

The third embodiment is shown in FIGS. 19 and 20. FIG. 19 corresponds generally to FIGS. 12 and 17 showing the second and modified second embodiments; FIG. 19 shows a single piece restrictor bobbin designed to function in essentially the same way as the second embodiment but in a smaller, 1 ml syringe. The differences of course include the fact that the overall component is smaller, having a total width from the tip of the barb 307 to the outer extremity of the protrusion 308 of approximately 0.2″. In the third embodiment, the bobbin is formed with a main arch 302 having first and second side walls 303, 304 respectively. Formed at the base of the side walls are laterally extending side plates 305, 306, respectively. The leading (distal) edge 304a of the second side wall is, as for the second embodiment, desiged to engage with the plunger in the same circumstances as described above in connection with the second embodiment. The second side wall 304 is also formed with a plunger barb 310 which has a somewhat different form to that of the second embodiment in that it is not pointed. This is a minor detail and the embodiment would work either with or without a pointed barb; in such a small component it was felt preferable not to form a point.

FIG. 20 shows the third embodiment including the syringe 350 with the bobbin 300 mounted on flange 355 of the plunger 352. FIG. 20 illustrates a state corresponding generally to that shown in FIG. 14 for the second embodiment, that is with the plunger 352 having been retracted and then depressed distally, causing the bobbin 300 to move distally from its starting position adjacent the undercut 353 at the proximal end of the barrel 351.

Assembly of the third embodiment is essentially the same as for the second embodiment.

A fourth embodiment of auto-disable syringe is shown in FIGS. 21 to 28. This embodiment aims to address the potential difficulty with blood sampling using the single component designs of embodiments two and three, as discussed above.

Referring firstly to FIG. 21, a single component restrictor bobbin 400 is shown which is similar in many respects to the bobbin 100 of the second embodiment. As with the second embodiment, the bobbin 400 of the fourth embodiment is intended to operate in a standard 5 ml syringe having an undercut at the proximal end of the barrel. The features of the bobbin 400 which are the same as the bobbin 100 of the second embodiment are labelled similarly except that the corresponding reference numerals commence at 400 instead of 100. These parts have identical functions to the corresponding parts in the second embodiment and thus require no further explanation. In addition, the bobbin 400 has a proximal extension arm 411 extending from the proximal end of the first side plate 405, adjacent the protrusion 408. At the proximal end of the extension arm 411 is a second protrusion 412 extending laterally in parallel to the protrusion 408. The second protrusion 412 has a bevelled distal outer edge 413 whilst its proximal outer edge is rounded. The second protrusion 412 extends laterally somewhat further than the first protrusion 408. The function of these additional features of the bobbin of the fourth embodiment will be explained below with reference to FIGS. 22 to 28 which show the syringe, with the bobbin 400 fitted, in various stages of operation.

FIG. 22 shows the syringe 450 in its shipped state with the plunger 452 in a fully depressed position. The bobbin 400 is in a non-rotated orientation at the proximal end of the syringe. The extension arm 411 projects proximally beyond the proximal end of the barrel 451.

The configuration of the bobbin 400 in the barrel 451 is shown in more detail in FIG. 23. The syringe plunger 452 is omitted from this drawing for clarity; the plunger should be understood to be located as shown in FIG. 22, with its flange 455 passing under the main arch 402 of the bobbin 400.

As may be seen in FIG. 23, the protrusion 408 is butted against the undercut 453 in the proximal end of the barrel 451. The arm 411 extends out of the mouth of the barrel 451 with a small clearance 417 between the second protrusion 412 and the end of the syringe barrel 451.

The sequence of operation of the fourth embodiment for withdrawing a blood sample will be described now with reference to FIGS. 24 to 28. As with the previous embodiments, the configuration of the bobbin 400 does not change as the syringe plunger 452 is initially withdrawn. FIG. 24 shows the plunger retracted a small distance as a user, having inserted the needle into a patient, draws back the plunger to check whether the needle is in a vein or not. If the needle is in a vein, then the user simply continues to draw blood into the syringe. If the needle is not in a vein, then the user returns the plunger to its most distal position and repositions the needle in the patient before making another attempt. FIG. 25 shows the plunger re-seated in its most distal position, whilst FIG. 26 provides further detail of the configuration of the bobbin 400 at this stage. As can be seen most clearly in FIG. 26, the bobbin 400 has been carried a small distance down the barrel, but is still retained in a non-rotated configuration due to the interaction of the protrusion 408 and the arm 411 and second protrusion 412 with the barrel undercut 453 and a tapered lead-in surface 454 at the most proximal end of the barrel. From this position it is possible to make a further attempt to withdraw blood, having repositioned the needle in the patient.

After a second attempt to draw blood, further distal movement of the plunger will force the second protrusion past the undercut 453 and into the syringe barrel, where its engagement with the barrel interior will force the bobbin 400 into a rotated position. FIG. 27 shows the plunger being moved distally and carrying the bobbin 400 past the barrel undercut 453. FIG. 28 shows the bobbin fully inside the barrel. In this embodiment, once this stage has been reached there is no longer any free play available in the plunger movement; the only permitted direction of movement is distally until the plunger is fully seated in its most distal position.

Assembly of the fourth embodiment is essentially the same as for the second and third embodiments.

The first to fourth embodiments each relate to syringes with plungers having a generally cruciform cross section, and to restrictor elements designed to fit onto such plungers. In a fifth embodiment shown in FIGS. 29 to 33, a 1 ml syringe having a circular cross section plunger shaft is fitted with a restrictor bobbin which functions in a similar way to the bobbin of the second and third embodiments.

In the fifth embodiment, the restrictor element or bobbin 500 comprises a main arch or main body 502 corresponding loosely to the main arch 302 of the third embodiment but having a generally semicircular section, which fits over the circular section plunger of the syringe. Unlike the other embodiments, there are no side plates and, instead, barbs extend directly from the main body 502. The component spans the barrel and is designed to wedge in the barrel to disable the syringe, as with the previous embodiments. However, as will be explained in detail below, the wedging rotation/camming action of the bobbin 500 is in a different sense to that of the first to fourth embodiments.

The main body 502 is stamped from 0.007″ stainless steel plate and formed into a shape which is somewhat more than a semicircle (about 270 degrees). Referring firstly to FIG. 29, the bobbin 500 is shown with its proximal end facing down and its distal end facing up. The centre of the arched main body 502 will be termed the “top” of the component and the two ends of the arch will be termed the “bottom” of the component (this terminology is consistent with the presentation of the device in FIGS. 32 and 33 where it is shown installed in a syringe). At the centre of the bobbin 500, on the distal edge is a gently curved cutaway 514, whose function will be explained later.

Extending proximally and slightly upwardly from the proximal edge, opposite the cutaway 514, is a pair of barrel engaging barbs 507. Between these barbs 507 is a slight depression 515 in the surface of the body 502, adjacent the proximal edge. At the bottom of the bobbin 500, in the region of each of the two ends of the arched body 502, is an area 516 of reduced dimension in the axial direction. Extending proximally and slightly inwardly from each of these areas 516 is a plunger engaging barb 510.

All of the above features may be seen in the different orientations of the component shown in FIGS. 29 to 31.

FIG. 32 shows the component fitted to a standard 1 ml syringe 550 having a circular section plunger shaft 552. As can be seen, the bobbin 500 is seated on the plunger shaft 552, situated between the plunger shaft and the syringe barrel 551. As with the previous embodiments, the barbs 507 are engaged with the interior surface of the barrel 551 (the upper part of the barrel has been removed in FIG. 32 for clarity) and oriented so as to resist proximal movement of the bobbin 500 with respect to the barrel 551, but to allow distal movement.

As with previous embodiments, the syringe is equipped with the standard feature of an undercut 553 at its proximal end. In the shipped state (which is not shown in the Figures), the bobbin 500 would be located such that the barbs 510 (unseen in FIGS. 32 and 33) are butted against the undercut 553. What also cannot be seen in FIGS. 32 and 33, but which will easily be understood from consideration of the previous embodiments, is that the barbs 510 on the bottom of the bobbin 500 are engaged with the plunger shaft.

In an operation to administer injected medication, the plunger 552 is first withdrawn from the position as shipped (as described above) to draw up medication. As with previous embodiments, there is no restriction to the plunger being withdrawn proximally, until the plunger head engages with the distal end of the bobbin 500. The next step is normally to expel air and excess medication from the syringe by partially depressing the plunger 552 in the distal direction. As this is done, the bobbin 500 is moved distally since the plunger engaging barbs 510 prevent movement of the plunger distally past the bobbin, in a way wihich is analogous to the first to third embodiments.

Again in a way analogous to the first to third embodiments, the bobbin 500 prevents further substantial proximal movement of the plunger at this point, but permits a small degree of proximal movement to allow for aspiration to check the position of the needle in the patient. FIG. 33 shows the “challenged” position of the bobbin 500 as the plunger is withdrawn proximally from the position shown in FIG. 32. The difference between the drawings is quite subtle, but it may be seen that in FIG. 32 there is a small clearance 517 between the bobbin 500 and the shaft of the plunger 552. This clearance is maintained in the non-rotated configuration of the bobbin 500 by the outer edge of the depression 515 bearing against the plunger; this also has the effect of keeping the barbs 510 engaged with the opposite side of the plunger. As the plunger is withdrawn, the small degree of resistence between the plunger barbs 510 and plunger causes the bobbin 500 to rotate in a sense which brings the bottom edge of the cutaway 514 into contact with the plunger shaft. It can be seen in FIG. 33 that the clearance 517 has disappeared at the distal end of the bobbin (at the cutaway 514) and the angle of the barrel-engaging barbs 507 has increased so that they are pointed more upwardly towards the barrel. The bottom edge of the cutaway 514 engages with the plunger such as to prevent further proximal movement of the plunger, but distal movement of the plunger will simply cause this wedged state to reverse as the bobbin rotates essentially about a notional axis drawn between the points of the barrel-engaging barbs 507.

One difference between this embodiment and the previous embodiments is that the bobbin does not tend to wedge across the barrel 551; that is to say, in the wedged position the bottom part of the bobbin is not necessarily engaged with the barrel. Wedging of the bobbin occurs between the inner surface of the un-shown upper part of the barrel (in FIGS. 32 and 33) and the upper surface of the plunger shaft. When force is applied to the plunger in the proximal direction, the camming of the bobbin will tend to cause the plunger to deform laterally and its bottom surface to bear against the barrel.

Whilst it would be possible to design the bottom part of the bobbin with rounded barrel engaging surfaces so that the bobbin always engages the barrel, and wedges across the barrel when in the “challenged” state, it has not been found necessary in this case. Similarly, it would be possible with the other embodiments for the protrusion 8, 108, 208, 308, 408 to be omitted (or even the entire first side plate 5, 105, etc.) and for all the wedging action of the bobbin to happen between plunger and barrel as in the fifth embodiment, though this has not been found to be so effective in the first to fourth embodiments.

Assembly of the fifth embodiment is very convenient: The stainless steel bobbin 500 is sufficiently elastic that it can easily be clipped over the plunger shaft, with the end regions 516 of the body 502 resiliently deforming to allow the bobbin to clip into place. Thereafter the bobbin will be retained on the plunger shaft during the assembly process, regardless of the orientation of the plunger. The plunger may be inserted into the barrel with the bobbin 500 installed on it at the most proximal end of the plunger; once the plunger is fully seated, a small tool may be used to push the bobbin 500 along the plunger and past the undercut, which will require small additional force. Alternatively the bobbin may simply be mounted on the plunger and the plunger then inserted, as with previous embodiments. The barbs 510 will prevent the bobbin 510 moving as the plunger is pushed in, though as with the other embodiments, it may be desirable in an automated process for a tool to be used in addition to ensure that the bobbin reains in a correct position as the plunger is inserted.

FIG. 34 shows a sixth embodiment of restrictor element deigned for use with a standard 1 ml syringe with a cruciform shaft (specifically a 1 ml “Omnifix”® syringe from B Braun. The sixth embodiment is similar in most repsects to the third embodiment and similar reference numbers will be used to designate similar parts, but with the number series commencing at 600.

As with the third embodiment, the component is formed with a central arch 602 with side plates 605 and 606 extending laterally from the end of each leg of the arch. Directed inwardly from one side of the arch is a proximally facing barb 610 whose function is to resist movement of the restrictor element in a proximal direction relative to the plunger shaft, when mounted on a plunger (i.e. to resist distal movement of the plunger with respect to the element).

As with the third embodiment, the component will initially sit against the barrel undercut until the plunger is moved distally, when the component is carried distally with the plunger. Subsequent proximal movement of the plunger is substantially prevented by engagement of the barb 607 with the interior of the barrel, though a degree of rotation about the barb 607 is possible as with the third embodiment.

The sixth embodiment is designed with a cutaway portion 611 at the top of the arch 602, to prevent the arch interfering with the barrel wall as the component rotates. Another difference from the third embodiment is that the side plates extend further in the longitudinal direction. This feature is to increase the security of the system when challenged; if extreme proximal force is applied to the plunger, the arch 602 may deform and allow the side plates, especially side plate 606, to rotate relative to the arch. The increased length of side plate 606 ensures that it will wedge across the space between the plunger and barrel.

A seventh embodiment of restrictor element is shown in FIG. 7, which is designed for use in a standard B Braun Omnifix® 5 ml syringe. The seventh embodiment is a single component restrictor element whose geometry does not cause it to rotate and wedge when a plunger on which it is mounted is withdrawn proximally. The basic shape of the element is similar to that of the seond embodiment as shown in FIG. 12: it comprises a main arch 702, in one limb of which is formed a barb 710, and side plates 705, 706 with a barb 707 being provided at the outer end of one of the plates. As with the second embodiment, the barbs 710 and 707 are for engaging the plunger and barrel respectively to ensure that the element is carried with the plunger when it is moved distally and that the element resists being moved proximally with respect to the barrel. The side plate 705, however, is formed with twin protrusions 708a, 708b which prevent the component rotating significantly when the plunger is moved proximally. The component therefore does not wedge against the plunger but allows the plunger to be moved proximally until the distal edges of the side plates 705, 706 contact the rear (proximal) face of the plunger head. One the plunger head contacts the component 700, the component will rotate about the barb 706, causing the barb to dig deeply into the barrel wall and preventing further substantial movement in a proximal direction, whilst also permitting a small amount of repeatable back and forth movement. The side plate 706 has an inclined distal edge 706a which helps ensure that the component cams/rotates as the rear face of the plunger engages with it.

The seventh embodiment does not prevent the administration of multiple doses of medicament with a combined total volume up to that of the syringe, as discussed above. This disadvantage may be outweighed by the fact that the seventh embodiment may be more suitable for withdrawal of bodily fluid, since there is no danger that initial distal movement of the plunger (e.g. for aspiration prior to withdrawal of fluid) will cause the syringe to be locked against further substantial proximal movement of the plunger (see discussion above).

1. A syringe comprising:
(a) a plunger including a plunger head and a shaft;
(b) a barrel;
(c) a restrictor bobbin engaged with the plunger shaft;
(d) the restrictor bobbin having a outer barb, tine, serration, edge or the like engaged with an interior surface of the barrel;
(e) the restrictor bobbin or a first part thereof being rotatable within the barrel;
(f) whereby movement of the plunger in a predetermined direction causes the restrictor bobbin, or said first part thereof, to rotate and wedge in the barrel.

2. A syringe as claimed in claim 1 wherein the said restrictor bobbin or said first part thereof wedges between the barrel and plunger.

3. A syringe as claimed in claim 1 wherein the said restrictor bobbin or said first part thereof substantially spans the barrel.

4. A syringe as claimed in any of claim 1 wherein the said predetermined direction is the proximal direction.

5. A syringe as claimed in claim 1 wherein the plunger shaft is substantially uniform over at least 70% of its length.

6. A syringe as claimed in claim 1 wherein the barrel inner surface is substantially uniform over substantially its entire usable extent, and is provided with an undercut at or adjacent the proximal end of its usable extent.

7. A syringe as claimed in claim 1 wherein rotation of the bobbin, or first part thereof, provides a predetermined degree of lost motion whereby the plunger may be cycled repeatedly over a predefined distance.

8. A syringe as claimed in claim 7 wherein the said predefined distance is between 1 and 25% of the usable extent of the syringe.

9. A syringe as claimed in claim 8 wherein the said predefined distance is between 5 and 15% of the usable extent of the syringe.

10. A syringe comprising:
(a) A plunger,
(b) A barrel,
(c) A restrictor bobbin, wherein the restrictor bobbin includes:-
A barrel locking formation or formations (e.g. an edge, barb, tine or serration) in engagement with the interior surface of the barrel and arranged to permit only unidirectional movement with respect to the barrel,
Any such barrel locking formation or formations being located on a first side only of the bobbin,
A plunger locking formation (e.g. an edge, barb, tine, serration) arranged to be brought into or out of engagement with the plunger by rotation of the bobbin.

11. A syringe comprising:
(a) A plunger,
(b) A barrel,
(c) A restrictor bobbin, wherein the restrictor bobbin includes:-
an arch formation mounted over the syringe plunger,
said arch formation and plunger being configured to allow limited relative rotation between the formation and the plunger,
said arch formation having on only one side thereof a barrel locking formation or formations (e.g. an edge, barb, tine or serration) in engagement with the interior surface of the barrel and arranged to permit only unidirectional movement with respect to the barrel,
an edge of said arch formation being engageable with the plunger on rotation of the bobbin thereby to restrict relative movement of the plunger and bobbin in a predetermined direction.

12. A restrictor element for use in an auto-disable syringe comprising a shaped plate with a central U-shaped portion adapted to be located on a syringe plunger shaft and first and second flanges extending from opposite sides of the central U-shaped portion, the element having a proximal and distal end and having:-
(a) an outer barb, tine or serration located on the said first flange at an edge remote from the central U-shaped portion and oriented such as to resist movement of the element in a proximal direction with respect to a syringe barrel in which it is installed, by penetrating a wall of the said barrel
(b) an inner barb, tine or serration located on an opposite side of the element to the said outer barb, tine or serration and oriented such as to resist movement of the element in a proximal direction with respect to a syringe plunger shaft on which it is installed;
(c) the second flange of the restrictor element being adapted not to penetrate the said barrel wall.

13. An auto-disable syringe comprising a standard syringe having only a single unitary restrictor element or bobbin fitted therein to provide an auto-disable function.

14. A syringe as claimed in claim 13 wherein unlimited repeated proximal and distal movement of the syringe plunger is possible over distance of between 1 and 25% of the full usable extent of the syringe.

15. A syringe as claimed in claim 14 wherein unlimited repeated proximal and distal movement of the syringe plunger is possible over distance of between 5 and 15% of the full usable extent of the syringe.

16. A method of assembling an auto disable syringe comprising mounting a single, unitary restrictor bobbin on the plunger shaft of a standard syringe and then inserting the plunger into the barrel of the said standard syringe.
